SEQUENCE LISTING (12 sequences)

SEQ ID NO: 1 - protein, 345 aa, Homo sapiens
YGITSPISLASPEEIDHIYTQKLIDAMKPFGVFEDEEELNHRLVVLGKLNNLVKEWISDVSESKNLPPSVVATVGGKIFTFGSYRLGVHTKGADIDALCVAPRHVERSDFFQSFFEKLKHQDGIRNLRAVEDAFVPVIKFEFDGIEIDLVFARLAIQTISDNLDLRDDSRLRSLDIRCIRSLNGCRVTDEILHLVPNKETFRLTLRAVKLWAKRRGIYSNMLGFLGGVSWAMLVARTCQLYPNAAASTLVHKFFLVFSKWEWPNPVLLKQSEESNLNLPVWDPRVNPSDRYHLMPIITPAYPQQNSTYNVSTSTRTVMVEEFKQGLAVTDEILQGKSDWSKLLEP

SEQ ID NO: 2 - protein, 345 aa, Drosophila melanogaster
LGMTSAISLAEPRPEDLQRTDELRGSLEPYNVFESQDELNHRMEILAKLNTLVKQWVKEISVSKNMPESAAEKLGGKIYTFGSYRLGVHHKGADIDALCVAPRNIERTDYFQSFFEVLKKQPEVTECRSVEEAFVPVIKMNFDGIEIDLLFARLSLKEIPDDFDLRDDNLLRNLDHRSVRSLNGCRVTDEILALVPNIENFRLALRTIKLWAKKHGIYSNSLGYFGGVTWAMLVARTCQLYPNAAAATLVHKFFLVFSRWKWPNPVLLKHPDNVNLRFQVWDPRVNASDRYHLMPIITPAYPQQNSTFNVSESTKKVILTEFNRGMNITDEIMLGRIPWERLFEA

SEQ ID NO: 3 - protein, 345 aa, Saccharomyces cerevisiae
FGITGPVSTVGATAAENKLNDSLIQELKKEGSFETEQETANRVQVLKILQELAQRFVYEVSKKKNMSDGMARDAGGKIFTYGSYRLGVHGPGSDIDTLVVVPKHVTREDFFTVFDSLLRERKELDEIAPVPDAFVPIIKIKFSGISIDLICARLDQPQVPLSLTLSDKNLLRNLDEKDLRALNGTRVTDEILELVPKPNVFRIALRAIKLWAQRRAVYANIFGFPGGVAWAMLVARICQLYPNACSAVILNRFFIILSEWNWPQPVILKPIEDGPLQVRVWNPKIYAQDRSHRMPVITPAYPSMCATHNITESTKKVILQEFVRGVQITNDIFSNKKSWANLFEK

SEQ ID NO: 4 - protein, 349 aa, Caenorhabditis elegans
LGVSQPISLAHPDSKDIAQTTLLIETLKKFGSYEPKEETEQRMEVLRNLNRLVKEWVKNVTAMKIPNGEGVNAGGKLFTFGSYRLGVHSSGADIDTLAVVPRHIDRSDFFTSFKEMLNNDPNVTELHGVEEAFVPVMKLKYSGVELDILFARLALKEVPDTQELSDDNLLRNLDQESVRSLNGCRVAEQLLKLVPRQKEFCVTLRAIKLWAKNHGIYSNSMGFFGGITWAILVARACQLYPNASPSRLVHRMFFIFSTWTWPHPVVLNEMNNDRNDIPTLCELVWDPRRKNTDRFHVMPIITPAFPEQNSTHNVTRSTATVIKNEICEALEICRDISEGKSKWTALFEE

SEQ ID NO: 5 - protein, 388 aa, Caenorhabditis elegans
RGFASPSPPTSLLSEPLSRMDVLSEKIWDYHNKVSQTDEMLQRKLHLRDMLYTAISPVFPLSGLYVVGSSLNGFGNNSSDMDLCLMITNKDLDQKNDAVVVLNLILSTLQYEKFVESQKLILAKVPILRINFAAPFDDITVDLNANNSVAIRNTHLLCYYSSYDWRVRPLVSVVKEWAKRKGINDANKSSFTSYSLVLMVIHFLQCGPTKVLPNLQQSYPNRFSNKVDVRTLNVTMALEEVADDIDQSLSEKTTLGELLIGFLDYYANEFNYDRDAISIRQGRRVERAALAVRPKIHSNSEGDKETPPPSSSASTSSIHNGGTPGIPMHHSISNPHFWRSQWRCVCIEEPFTNSNTAHSIYDEMVFEAIKKAFREAHGELQHNHDLDK

SEQ ID NO: 6 - protein, 387 aa, Caenorhabditis elegans
GFASPSPPTSLLSEPLSRMDVLSEKIWDYHNKVSQTDEMLQRKLHLRDMLYTAISPVFPLSGLYVVGSSLNGFGNNSSDMDLCLMITNKDLDQKNDAVVVLNLILSTLQYEKFVESQKLILAKVPILRINFAAPFDDITVDLNANNSVAIRNTHLLCYYSSYDWRVRPLVSVVKEWAKRKGINDANKSSFTSYSLVLMVIHFLQCGPTKVLPNLQQSYPNRFSNKVDVRTLNVTMALEEVADDIDQSLSEKTTLGELLIGFLDYYANEFNYDRDAISIRQGRRVERAALAVRPKIHSNSEGDKETPPPSSSASTSSIHNGGTPGIPMHHSISNPHFWRSQWRCVCIEEPFTNSNTAHSIYDEMVFEAIKKAFREAHGELQHNHDLDK

SEQ ID NO: 7 - protein, 336 aa, Mus musculus
EIPLLEPREITLPEAKDKLSQQILELFETCQQQASDLKKKELCRAQLQREIQLLFPQSRLFLVGSSLNGFGARSSDGDLCLVVKEEPCFFQVNQKTEARHILTLVHKHFCTRLSGYIERPQLIRAKVPIVKFRDKVSCVEFDLNVNNTVGIRNTFLLRTYAYLENRVRPLVLVIKKWASHHDINDASRGTLSSYSLVLMVLHYLQTLPEPILPSLQKIYPESFSTSVQLHLVHHAPCNVPPYLSKNESSLGDLLLGFLKYYATEFDWNTQMISVREAKAIPRPDDMEWRNKYICVEEPFDGTNTARAVHEKQKFDMIKDQFLKSWQRLKNKRDLNS

SEQ ID NO: 8 - protein, 336 aa, Homo sapiens
EIAFLEPREITLPEAKDKLSQQILELFETCQQQISDLKKKELCRTQLQREIQLLFPQSRLFLVGSSLNGFGTRSSDGDLCLVVKEEPCFFQVNQKTEARHILTLVHKHFCTRLSGYIERPQLIRAKVPIVKFRDKVSCVEFDLNVNNIVGIRNTFLLRTYAYLENRVRPLVLVIKKWASHHQINDASRGTLSSYSLVLMVLHYLQTLPEPILPSLQKIYPESFSPAIQLHLVHQAPCNVPPYLSKNESNLGDLLLGFLKYYATEFDWNSQMISVREAKAIPRPDGIEWRNKYICVEEPFDGTNTARAVHEKQKFDMIKDQFLKSWHRLKNKRDLNS

SEQ ID NO: 9 - protein, 357 aa, Caenorhabditis elegans
IAGSVEKFVNSITKKSFNSVKQLSKLAWDHYLGNAQPDFVFLKKMEARQKLFSEIKKLFPDTEIKLQTTGSTVNGCGSFNSDMDLCLCFPTNGYKGQVCDDFHCDRNYSTKILRKIDKAFRRSHWSHPLKKIIKTMQLVPAKVPIVKMILNGEFDGIEVDINVNNIAGIYNSHLIHYYSLTDARLPALALLVKHWAMVTGINNAQDGFLNSYTTILLVVHYLQCGVTPAVIPNLQYLFPHKFDRKLPLNELLLFGDIADKLPTSPPNTWSLGELLIGFFQYYNEFDFTNFGFSIRSGQVIPRENLPRDLINSPIVVEEPFDAINTARTVRDVSHMKSIKSAFRCAVQIISSNKNFTM

SEQ ID NO: 10 - protein, 332 aa, Arabidopsis thaliana
QKARMVKMYMACRNDIHRYDATFIAIYKSLIPAEEELEKQRQLMAHLENLVAKEWPHAKLYLYGSCANSFGFPKSDIDVCLAIEGDDINKSEMLLKLAEILESDNLQNVQALTRARVPIVKLMDPVTGISCDICINNVLAVVNTKLLRDYAQIDVRLRQLAFIVKHWAKSRRVNETYQGTLSSYAYVLMCIHFLQQRRPPILPCLQEMEPTYSVRVDNIRCTYFDNVDRLRNFGSNNRETIAELVWGFFNYWAYAHDYAYNVVSVRTGSILGKREKDWTRRVGNDRHLICIEDPFETSHDLGRVVDKFSIRVLREEFERAARIMHQDPNPCA

SEQ ID NO: 11 - protein, 349 aa, Homo sapiens
PEDFKRIQLEPLPPLTPKFLNILDQVCIQCYKDFSPTIIEDQAREHIRQNLESFIRQDFPGTKLSLFGSSKNGFGFKQSDLDVCMTINGLETAEGLDCVRTIEELARVLRKHSGLRNILPITTAKVPIVKFFHLRSGLEVDISLYNTLALHNTRLLSAYSAIDPRVKYLCYTMKVFTKMCDIGDASRGSLSSYAYTLMVLYFLQQRNPPVIPVLQEIYKGEKKPEIFVDGWNIYFFDQIDELPTYWSECGKNTESVGQLWLGLLRFYTEEFDFKEHVISIRRKSLLTTFKKQWTSKYIVIEDPFDLNHNLGAGLSRKMTNFIMKAFINGRRVFGIPVKGFPKDYPSKME

SEQ ID NO: 12 - protein, 345 aa, Bos taurus
YGITSPISLAAPKETDCLLTQKLVETLKPFGVFEEEEELQRRILILGKLNNLVKEWIREISESKNLPQSVIENVGGKIFTFGSYRLGVHTKGADIDALCVAPRHVDRSDFFTSFYDKLKLQEEVKDLRAVEEAFVPVIKLCFDGIEIDILFARLALQTIPEDLDLRDDSLLKNLDIRCIRSLNGCRVTDEILHLVPNIDNFRLTLRAIKLWAKRHNIYSNILGFLGGVSWAMLVARTCQLYPNAIASTLVHKFFLVFSKWEWPNPVLLKQPEECNLNLPVWDPRVNPSDRYHLMPIITPAYPQQNSTYNVSVSTRMVMVEEFKQGLAITDEILLSKAEWSKLFEA

(19) United States
(12) Patent Application Publication
(10) Pub. No.: US 2004/0235001 A1
(43) Pub. Date: Nov. 25, 2004

(54) Regulatory poly(A) polymerase and uses thereof

(76) Inventors: Liaoteng Wang, Madison, WI (US); Marvin P. Wickens, Madison, WI (US); Judith E. Kimble, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 10/665,797

(22) Filed: Sep. 18, 2003

Related U.S. Application Data
(60) Provisional application No. 60/411,685, filed on Sep. 18, 2002.

(51) Int. Cl. C12Q 1/68; C07H 21/04; C12P 19/34; C12N 9/22

(52) U.S. Cl. 435/6; 435/69.1; 435/91.2; 435/199; 435/320.1; 435/325; 536/23.2

(57) ABSTRACT

An isolated preparation of a regulatory poly(A) polymerase (PAP), wherein the polymerase comprises both a catalytic subunit and an RNA-binding subunit is disclosed.

### CROSS-REFERENCE TO RELATED APPLICATION

This application claims priority to provisional serial no. 60/411,685, filed Sep. 18, 2003.

### STATEMENT REGARDING FEDERALLY SPONSORED RESEARCH OR DEVELOPMENT

This invention was made with United States government support awarded by the following agency: NIH GM031892. The United States has certain rights in this invention.

### BACKGROUND OF THE INVENTION

mRNAs are exquisitely controlled in eukaryotic cells. Regulated mRNA stability, translation and localization are essential for early development, cell growth, homeostasis, neuronal plasticity (Wickens, M., et al., in Translational Control of Gene Expression, eds. Hershey, J. W. B., et al., Cold Spring Harbor Laboratory Press, Cold Spring Harbor, N.Y., pp. 295-370, 2000; Dever, T. E., Cell 108:545-556, 2002; Job, C. and Eberwine, J., Nat. Rev. Neuro. 2:889-898, 2001; Martin, K. C., et al., Curr. Opin. Neuro. 10:587-592, 2000; Richter, J. D., in Translational Control of Gene Expression, eds. Hershey, J. W. B., et al., Cold Spring Harbor Laboratory Press, Cold Spring Harbor, N.Y., pp. 785-805, 2000; Bushell, M. and Sarnow, P., J. Cell Biol. 158:395-399, 2002; Patil, C. and Walter, P., Curr. Opin. Cell Biol. 13:349-355, 2001). A tract of adenosine residues added post-transcriptionally to the 3′ end of the mRNA-poly(A)—is a hallmark of mRNAs, and a plexus of control (Wickens, M., et al., supra, 2000; Jacobson, A., in Translational Control, eds. Hershey, J. W., et al., Cold Spring Harbor Laboratory Press, Plainview, N.Y., pp. 451-480,1996; Jacobson, A. and Peltz, S. W., Ann. Rev. Biochem. 65:693-739, 1996; Sachs, A. B., et al., Cell 89:831-838, 1997). In the nucleus, poly(A) addition is linked to cleavage of the pre-mRNA. The machinery involved communicates with splicing and transcription factors (Proudfoot, N. J., et al., Cell 108:501-512, 2002; Zhao, J., et al., Micro. Mol. Biol. Rev. 63:405-445,1999; Hirose, Y. and Manley, J. L., Genes Dev. 14:1415-1429, 2000; McCracken, S., etal., Nature 385:357-361, 1997; Shatkin, A. J. and Manley, J. L., Nat. Struct. Biol. 7:838-842, 2000; Dower, K. and Rosbash, M., RNA 8:686-697, 2002) and is regulated by DNA damage, mitosis, and differentiation (Hirose, Y. and Manley, J. L., supra, 2000; Minvielle-Sebastia, L. and Keller, W., Curr. Biol. 11:352-357,1999; Colgan, D. F. and Manley, J. L., Genes Dev. 11:2755-2766,1997; Wahle, E. and Ruegsegger, U., FEMS Microbiol. Rev. 23:277-295,1999). Shortening of the poly(A) tail in the cytoplasm can trigger translational repression and mRNA decay, while lengthening can cause translational activation and mRNA stabilization (Wickens, M., et al., supra, 2000; Richter, J. D., supra, 2000; Jacobson, A. and Peltz, S. W., supra, 1996).

mRNAs emerge from the nucleus with a long tail. In the default state, this tail is shortened in the cytoplasm at a slow rate. However, RNA-protein complexes formed in the 3′UTR (untranslated region) can accelerate deadenylation. Conversely, other 3′UTRs recruit factors that add poly(A) in the cytoplasm at characteristic times, leading to a net increase in poly(A) length (Wickens, M., et al., supra, 2000; Richter, J. D., supra, 2000). These events are best characterized in early embryos, and contribute to pattern formation and cell cycle control (Wickens, M., et al., supra, 2000; Richter, J. D., supra, 2000). In neuronal cells, regulated cytoplasmic polyadenylation at synapses controls local translation, and may be essential for learning and memory (Job, C. and Eberwine, J., supra, 2000; Martin, K. C., et al., supra, 2000; Richter, J. D., supra, 2000). However, the enzymes responsible for cytoplasmic polyadenylation in neurons and other somatic cells have not been identified.

The enzyme that adds poly(A) in the nucleus, a “canonical” eukaryotic PAP (poly A polymerase), is highly conserved, and adds poly(A) one nucleotide at a time (Raabe, T., et al., Nature 353:229-234, 1991; Wahle, E., et al., EMBO J. 10:4251-4257, 1991; Bardwell, V. J., et al., Mol. Cell. Biol. 10:846-849,1990; Doublie, S., EMBO J. 19:4193-4203, 2000; Bard, J., et al., Science 289:1346-1349, 2000). The PAP is a poor RNA binding protein and so is relatively inactive on its own (Wahle, E. and Ruegsegger, U., FEMS Micro. Rev. 23:277-295,1999; Bienroth, S., et al., EMBO J. 12:585-594, 1993). Although purified PAP acts as a monomer, the nuclear PAP assembles in vivo into a large multiprotein complex that recognizes specific sequences in the pre-mRNA. This complex cleaves the pre-mRNA to generate a new 3′ hydroxyl group, to which the PAP then adds poly(A).

The C. elegans gld-2 gene was identified initially through its specific effects on germline development (Kadyk, L. C. and Kimble, J., Development 125:1803-1813, 1998). The GLD-2 protein is cytoplasmic (Wang, L., et al., supra, 2002). GLD-2 is a member of the nucleotidyl transferase superfamily, which also includes canonical nuclear PAPs, as well as CCA adding enzymes, DNA polymerases and 2′-5′ oligo(A) synthetases (Aravind, L. and Koonin, E. V., Nucl. Acids Res. 27:1609-1618, 1999); however, GLD-2 diverges substantially from them throughout its length (Wang, L., et al., supra, 2002). It appears to lack the C-terminal RNA-binding motifs required for activity of nuclear PAPs (Doublie, S., supra, 2000; Bard, J., et al., supra, 2000; Aravind, L. and Koonin, E. V., supra, 1999; Zhelkovsky, A., et al., Mol. Cell. Biol. 18:5942,1998; Raabe, T., et al., Mol. Cell. Biol. 14:2946-2957,1994; Martin, G. and Keller, W., EMBO J. 15:2593-2603,1996). Distinct RNA-binding proteins stimulate its polyadenylation activity in vitro (Wang, L., et al., supra, 2002). For example, GLD-3, a member of the KH protein family (Eckmann, C. R., et al., Dev. Cell 3:697-710, 2002), binds to GLD-2 and stimulates its polyadenylation activity in vitro (Wang, L., et al., supra, 2002). Proteins that are similar in sequence to the GLD-2 catalytic subunit have been identified in S. pombe, possess poly(A) polymerase activity and reside in the cytoplasm (Wang, L., et al., Nature 419:312-316, 2002; Saitoh, S., et al., Cell 109:563-573, 2002; Read, R. L., et al., Proc. Natl. Acad. Sci. USA 99:12079-12084, 2002; Keller, W. and Martin, G., Nature 419:267-268, 1996). They may have RNA binding partners that are required for full activity.

We disclose below that GLD-2 is the catalytic subunit of a novel, heterodimeric PAP involved in cytoplasmic polyadenylation (Wang, L., et al., supra, 2002). We refer herein to the GLD-2-related subunits as the “catalytic subunits” of the regulatory PAP (rPAP) complexes, to the RNA-binding (e.g., GLD-3) subunits as the “RNA-binding subunits,” and to the complexes (e.g., GLD-2/GLD-3) as “rPAP complex” or an “rPAP.” In this model, the RNA-binding subunit recruits the cytoplasmic rPAP subunit to the mRNA. The specificity of the RNA binding proteins results in regulation of a specific subset of mRNAs, and provides versatility in control. Thus the catalytic subunit, relatively inactive on its own, acquires activity and sequence specificity by recruitment to its substrate.

### BRIEF SUMMARY OF THE INVENTION

We disclose herein a new family of enzymes that regulate mRNAs in eukaryotes, the regulatory poly(A) polymerases (rPAPs). rPAPs add poly(A) to mRNAs in the cytoplasm, increasing their translational activity and stability. As a result, they increase the amount of protein produced from specific mRNAs. We refer to these polymerases as “rPAPs.” The rPAP polymerases comprise a catalytic subunit and an RNA-binding subunit. The catalytic subunit is either GLD-2 or a GLD-2 homolog.

In one embodiment, the present invention is an isolated preparation of an rPAP polymerase or an isolated preparation of a catalytic subunit of the polymerase. In a preferred form of the present invention, the polymerase or the catalytic portion is derived from the human GLD-2 gene. In another embodiment, the present invention is a polynucleotide encoding an rPAP or a subunit of an rPAP. Preferably, the polynucleotide encodes a catalytic subunit of human origin, most preferably, Hs-1 (BAC04629) or a variant that still retains catalytic activity.

In another embodiment, the present invention allows one to identify from primary sequence those candidate proteins that actually are catalytic subunits of rPAPs (GLD-2 homologs). One would compare the sequence of the candidate polymerase with the canonical sequence described below. Preferably, one would then subject a protein or peptide to a functional analysis.

In another embodiment of the present invention, one would subject a protein or peptide directly to a functional analysis.

Another embodiment of the present invention allows one to identify molecules that increase or decrease the activity of rPAPs. These molecules are envisioned to be useful in multiple clinical and biological contexts including cancer chemotherapy, manipulation of stem cell populations, enhancing learning and memory, and fertility. In a preferred embodiment, one would expose a candidate molecule to an rPAP or the catalytic portion of an rPAP and determine whether the candidate molecule increases or decreases polymerase activity.

Other embodiments, features and objects of the present invention will become apparent after review of the specification, claims and drawings.

### BRIEF DESCRIPTION OF THE SEVERAL VIEWS OF THE DRAWINGS

The patent or application file contains at least one drawing executed in color. Copies of this patent or patent application publication with color drawings will be provided by the Office upon request and payment of the necessary fee.

FIG. 1. The gld-2 gene and its transcripts. FIG. 1a: gld-2 exon/intron structure. Exons, open boxes, introns, thin lines. Colour coding as in FIG. 2. FIG. 1b, top, probes (see Methods). Bottom, northern blots of poly(A)+ RNAs from mixed stage wild-type 9WT) animals or from glp-1 mutant adults with no germ line (−GL). Size markers in kb. Arrows, gld-2 transcripts. Sizes of gld-2 transcripts on northern blots (4.7 kb, 4.6 kb and 4.0 kb) correspond to sizes predicted by cDNA analyses (4,533 nt, 4,273 nt and 3,691 nt, excluding the poly(A) tail). FIG. 4c: developmental expression of gld-2 mRNA. Northern blot of poly(A)+ RNAs from staged animals. E, embryo; L1-L4, first-fourth larval stage; A, adult; mixed, mixed stages. Above, 5′ probe, FIG. 1b; below, loading control. FIGS. 1d-g: In situ hybridization of dissected germ lines. FIGS. 1d-f, 5′ probe, FIG. 1b; open arrowhead, distal end of germ line. FIG. 1d: Germ line of wild-type hermaphrodite adult. FIG. 1e: Germ line of wild-type male adult; sp, spermatogenesis; WT, wild-type; FIG. 1f: germ line of gld-2(q497) homozygous mutant adult; FIG. 1g: germ line of wild-type hermaphrodite adult, probed with sense strand of cDNA fragment covering exons 2-8 (5′ probe, 1b).

FIG. 2. GLD-2 belongs to the polβ nucleotidyltransferase superfamily. FIG. 2a: The polβ superfamily (adapted from Kadyk and Kimble, J., supra, 1998). Small color-coded circles represent sub-families; grey circle shows Group 2 families. Most important here are the eukaryotic TRF, TRF4/5-related proteins; PAP, eukaryotic poly(A) polymerase; and 2′-5′A, 2′-5′ oligoA synthetase. FIG. 2b-d: Color coding of domains based on crystal structures of bovine and yeast PAPs (Martin, G., et al., supra, 2000; Bard, J., et al., supra, 2000). Gold, catalytic domain; blue, central domain; violet, RRM domain. FIG. 2b: GLD-2 and PAP domains compared. Drosophila (Dm), human (Hs) and yeast (Sc). GLD-2 domains identified by pfam search (Bateman, A., et al., Nucleic Acids Res. 30:276-280, 2002). Color-coded regions based on crystal structures of bovine PAP and yeast PAP (Martin, G., et al., supra, 2000; Bard, J., et al., supra, 2000). FIG. 2c: Bovine PAP 3-D structure, with key residues shown in stick form (adapted from Martin, G., et al., supra, 2000; Bard, J., et al., supra, 2000). Created by Rasmol based on pdb file 1F5A (for bovine PAP). FIG. 2d: Amino acid sequence alignment of GLD-2 and PAP core regions based on ClustalW program (Thompson, J. D., et al., Nucleic Acids Res. 22:4673-4680, 1994) and polβ superfamily analyses (Gough, J., et al., J. Mol. Biol. 313:903-919, 2001). Mutants designated below. Red, catalytic residues; green, required for ATP binding. FIG. 2e: Unrooted tree of GLD-2 and homologs, created with PHYLIP program (Felsenstein, J., Phylogeny Interference Package, Department of Genetics, University of Washington, Seattle, Wash., 1993), based on ClustalW alignment using parsimony. Species are: Ce, C. elegans; Dm, Drosophila; Hs, human; Mm, mouse; Os, rice; Sp, S. pombe; At, Arabidopsis. Only homologs with E-value less than 1e⁻¹⁰.in the first PSI bist were used; tree was built using the catalytic and central domain sequences as in 2d. **, Cid1 and GLD-2 both function in cell cycle control; functions of others are unknown.

FIG. 3. The GLD-2 protein. FIG. 3a: Western blot of proteins extracted from wild-type embryos (E), larval stages (L1-L4), and adults (A) (lanes 1-5) and from gld-2(h292) homozygous adults (lane 6), gld-2(q497/+) heterozygous adults (lane 7, and gld-2(q497) homozygous adults (lane 8). Top, protein detected using antibodies to N-terminal region of germline-specific protein. Bottom, loading control was a band detected using pre-immune serum. FIG. 3b: GLD-2 protein is cytoplasmic in the germ line. Extruded adult hermaphrodite germ line; GLD-2 protein is abundant in pachytene region and persists in oocytes. Magnified view shows lack of GLD-2 in nuclei (arrowheads) and presence of GLD-2 in granular form (arrow). FIG. 3c: GLD-2 protein is associated with P granules in early embryos. Embryos stained with antibody to a P granule marker, PGL-1 (Kyriakopoulou C. B., et al., supra, 2001), to GLD-2, and to a nuclear pore antigen. Top, late P0 embryo, GLD-2 co-localizes with P granules; second panel, 28-cell embryo, P4 shown by arrowhead; third panel, ˜100 cell embryo, germline precursor cells, Z2 and z3, shown by arrows; bottom, magnified view of a blastomere to show PGL-1 and GLD-2 co-localization (arrows). FIG. 3d-e: Transgenic strain AZ212. Left, Nomarski image; right, nuclei seen via histone::GFP marker. FIG. 3d: Mock-injected control, 14 cells. FIG. 3e: gld-2(RNAi).

FIG. 4. GLD-2/GLD-3 is a novel poly(A) polymerase. FIG. 4a: a. Left, GLD-2 deletion constructs used in yeast two-hybrid assays to map minimal region for GLD-3 interaction. Right, both filter assays (LacZ) and growth assays (HIS) were used to monitor the interaction. FIG. 4b: GLD-2 fragments are expressed at similar level. Western blot, Δ2 and Δ7 fragments as in FIG. 4a. FIG. 4c: Nucleotidyltransferase assay. Incorporation of ³²P-ATP measure in reticulocyte lysates programmed with plasmids encoding GLD-2, GLD-3 or variants. Data are reported as cpm, not molar quantities, because ATP concentration in lysate is not known. The lysate exhibits a background incorporation (10,000 cpm) independent of GLD-encoding plasmids which has been subtracted here. FIG. 4d: Poly(A) polymerase assay. Reaction products analyzed on a 12% sequencing gel and autoradiography. Left, shorter exposure; right, longer exposure. Below, SDS-PAGE, Western blot showing that proteins used were expressed at a similar level Ml, D608A; M2, E875K; C₃₅A₁₀, substrate; bPAP, bovine poly(A) polymerase.

FIG. 5. Relationship of GLD-2/GLD-3 enzyme to classical PAPs. Left, architecture of classical PAPs. Right, predicted architecture of GLD-2/GLD-3. Domains are color coded as in FIG. 2. gld-2(RNAi).

FIG. 6. Strategy of the tethered function assay. (A) Model: GLD-2 functions as a heterodimer in which GLD-2 contains the PAP active site, and GLD-3 provides RNA-binding specificity. Test: GLD-2 protein was tethered to a reporter mRNA using an unrelated RNA binding protein, MS2 coat protein fused to the N terminus of GLD-2. (B) The assay exploits two reporter mRNAs. The luciferase mRNA lacks poly(A) and contains MS2 binding sites; the β-galactosidase lacking MS2 sites is used as a control.

FIG. 7. MS2-GLD-2 stimulates translation and polyadenylation of the luciferase reporter RNA. (A) MS2/GLD-2 stimulates translation of the luciferase reporter but not the β-galactosidase reporter. (B) Western blotting. Oocytes were injected with an mRNA encoding a fusion protein and collected after 6-hour incubation at room temperature. The oocytes were lysed in PBS buffer containing protease inhibitors and whole cell extract equivalent to three oocytes was loaded onto each lane. MS2-fusion proteins were recognized by anti-MS2 antibody (3H4 antibody, from M. Kiledjian lab). (C) [α-³²P]UTP radiolabeled luciferase mRNA was injected into oocytes expressing the fusion proteins indicated. Oocytes were collected either immediately after reporter RNA injection (Oh) or after 16-hour incubation (16 h). RNA was extracted and poly(A)⁺ mRNA was separated from poly(A)⁻ RNA using oligo(dT) resin. RNA that bound the oligo(dT) resin is indicated as oligo (dT) and RNA that did not bind the oligo(dT)⁺ resin is indicated as oligo(dT)⁻.

FIG. 8. The minimal region required for activity corresponds to the catalytic and central domains. (A) Depiction of the truncated GLD-2 proteins. Gray boxes indicate the catalytic domains and black boxes indicate the central domains. Asp 608 corresponds to one of the three aspartate residues conserved in the β-nucleotidyl transferase family. (B) The tethered function assay was performed as in FIG. 6B. The active site mutant of GLD-2 (Δ1(608A)) was used as a negative control. (C) Western blotting was performed as in FIG. 7B except that HA-tag antibody was used to recognize proteins.

FIG. 9. GLD-related sequences and candidate PAPs. (A) Beta-nucleotidyl transferase super family. A variety of enzyme activities reside in proteins within the beta-nucleotidyl transferase super family. These include not only GLD-2 (and its relatives, TRF4 and TRF5), but CCA-adding enzymes (CCA), 2′-5′oligo(A) synthetases (2′-5′A). Terminal transferases, the polX polymerases, and enzymes that transfer nucleotidyl groups to other non-nucleic acid compounds. (B) Dendrogram representing the twenty sequences most similar to GLD-2. Sequences tested for PAP activity in our experiments are in boxes. Black boxes, sequences that possess PAP activity; white boxes with an “X,” sequences that were inactive as tethered proteins; grey boxes, sequences shown elsewhere to be cytoplasmic PAPs. (C) Domain structure and similarity among the GLD-2 proteins tested. See text for details.

FIG. 10. Identification of new poly(A) polymerases. (A) The assays were performed as in FIG. 6B. MS2/GLD-2 and MS2/GLD-2(D608) were used as controls. (B) Western blotting was performed as in FIG. 7B using HA-tag antibody. (C) Radiolabeled luciferase mRNA injection and RNA preparation were performed as in FIG. 7C. Luciferase RNAs from oocytes expressing either Mm1 or Hs1 were retained to oligo(dT) resin after 16-hour incubation.

FIG. 11. Amino acid sequence alignments of putative PAPs. (A) Ribbon diagram of bovine PAP 3D structure in complex with 3′dATP (gray molecule in the center), with key residues shown in stick form. Created by Rasmol based on PDB file 1F5A (for bovine PAP, Doublie, S., EMBO J. 19:4193-4203, 2000). (B) Multiple sequence alignment of the putative catalytic region of proteins tested in the tethered assay. Color-coding of amino acids and regions used the same scheme as in (A). D608, the 608th residue (aspartate) in C. elegans GLD-2 that is essential for activity (Wang, L., et al., supra, 2002).

FIG. 12. GIP-1 stimulates activity of GLD-2. Assays as in FIG. 4D. Lane 1, GLD-2 only; Lane 2, point mutation disrupting active site (D608A); lane 3, point mutation disurpting interaction with GLD-3 (E875K); Lane 4, GIP-1 only; Lane 5, GLD-2 plus GIP-1.

### DETAILED DESCRIPTION OF THE INVENTION

### A. In General

Messenger RNA regulation is a critical mode of controlling gene expression. Regulation of mRNA stability and translation is linked to controls of poly(A) tail length (Richter, J. D., Translational Control of Gene Expression [eds Sonenberg, N., et al.,] pp. 785-805, 2000; Wickens, M., et al., Translational Control of Gene Expression [eds Sonenberg, N., et al.] pp. 295-370, 2000). Poly(A) lengthening can stabilize and translationally activate mRNAs, whereas poly(A) removal can trigger degradation and translational repression. Germline granules (for example, polar granules in flies, P granules in worms) are ribonucleoprotein particles implicated in translational control (Seydoux, G. and Strome, S., Development 126:3275-3283, 1999).

The present invention concerns our observation that a specific interaction between the C. elegans protein GLD-2 and another germline regulator, GLD-3, occurs in yeast two hybrid screens and that the GLD-2/GLD-3 interaction appears to be important for development. We believe that GLD-2 is the catalytic subunit of a cytoplasmic regulatory poly(A) polymerase and that combined with its GLD-3 partner it forms both the catalytic and RNA binding subunits of a novel heterdimeric poly(A) polymerase.

GLD-3 is not the only native RNA-binding protein partner of GLD-2. We have identified two other putative RNA-binding proteins, GIP-1 (GLD-2 interacting protein) and GIP-2, that bind to GLD-2. We suspect that these proteins also stimulate GLD-2's activity on a selected RNA.

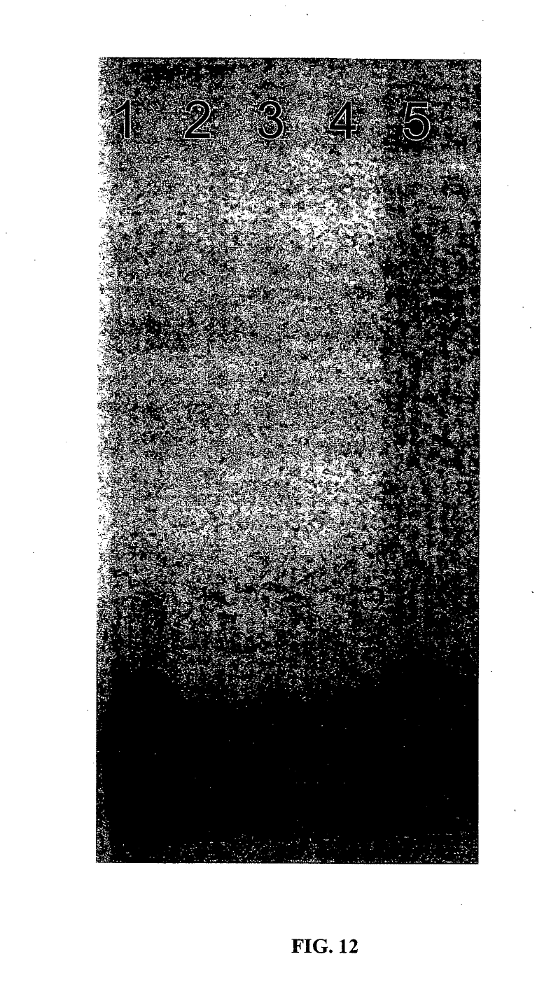

Referring to FIG. 12, C. elegans GIP-1 is transcript designation R119.7; C. elegans GIP-2 is transcript designation R114.7. GIP-1 stimulates GLD-2 activity in vitro, as shown in FIG. 12. We suspect that GIP-2 does as well. Human homologs of these sequences can be identified by sequence inspection, and include XP166699, MD39257, and KIAA0731.

We have defined a regulatory poly(A) polymerase (rPAP) as comprising a catalytic and an RNA-binding subunit. The catalytic subunit is either GLD-2 or a homolog of GLD-2. Two homologs of GLD-2 are identified below (Hs-1 and Mm-1).

(Applicants note that hRPAP1 is alternatively referred to as Hs-1 in this text. mRPAP is alternatively referred to as Mm-1.)

By “catalytic subunit” of an rPAP or “homolog of GLD-2” we mean to include proteins with the particular enzymatic activity displayed by GLD-2 in the Examples below. Specifically, this subunit will have the following characteristics:

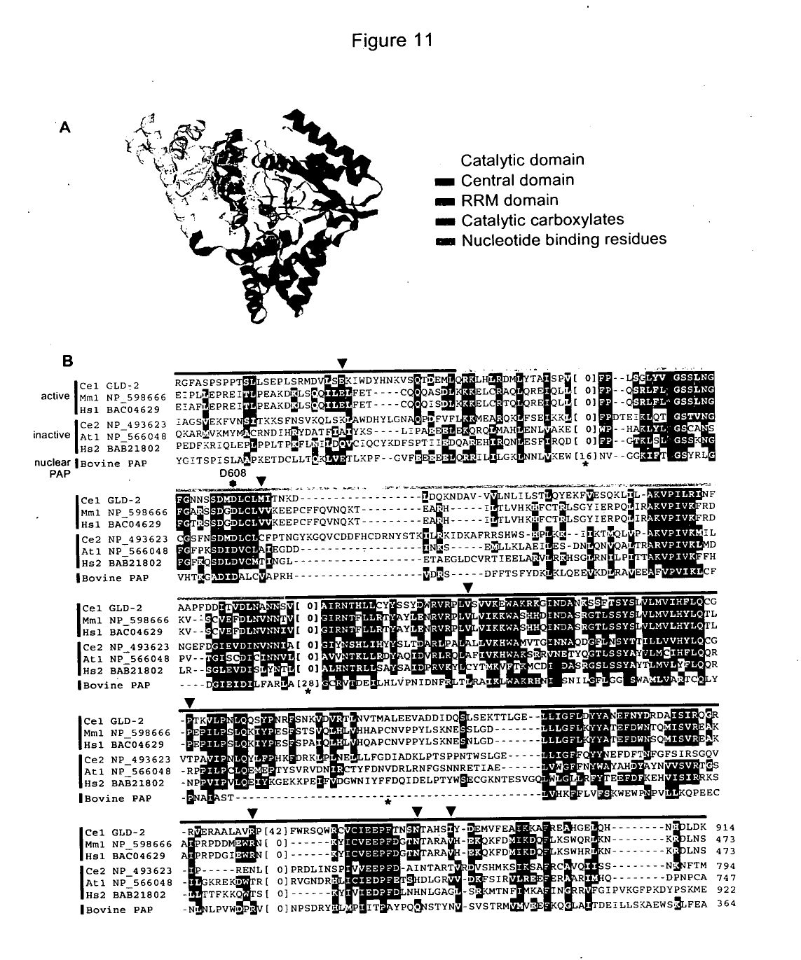

(1) They are related in sequence to GLD-2 throughout their catalytic domain. The candidate sequence must possess the three catalytic carboxylates, and the residues required for positioning the nucleotide. These residues must be in the order and with the approximate spacing as that presented in FIG. 11.

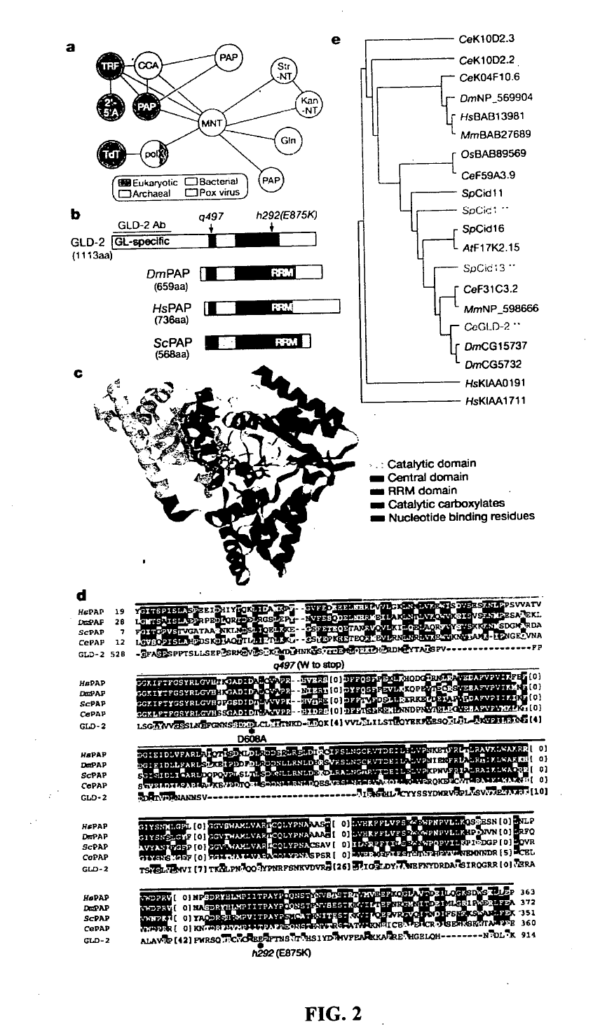

(2) They are not canonical nuclear PAPs (e.g., as depicted in FIG. 2).

(3) Their activity is stimulated by recruitment to an RNA via a designed RNA-protein interaction. We demonstrate this property below using yeast (TF-5), worm, mouse (Mm1 or mouse rPAP [NP_{—}598666]), frog, and human (Hs-1 or human rPAP [BAC04629]) enzymes.

We have identified GLD-3, GIP-1 and GIP-2 as RNA-binding proteins. Other appropriate RNA-binding proteins may be identified. The RNA-binding subunit recruits the catalytic subunit to an RNA, and thereby stimulates its activity. The RNA-protein interaction may be between the RNA-binding partner and a natural mRNA substrate or one contrived for that purpose. For example, GLD-3 recruits GLD-2 to a synthetic oligo(C) RNA. A suitable RNA-binding subunit would bind to the catalytic subunit of an rPAP and increase the polymerase activity of the catalytic subunit. The RNA-binding protein would also have to bind to the desired mRNA.

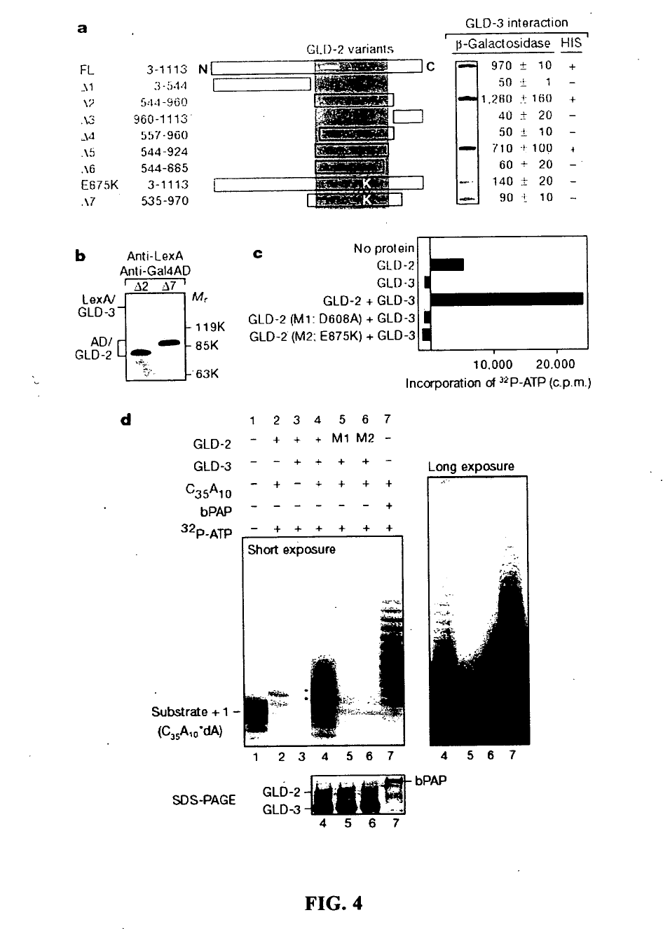

Proteins that interact physically with GLD-2 and its homologs from other species can be identified by methods known to those skilled in the art. Among all the proteins that interact, those of interest here will stimulate the activity of the catalytic subunit (e.g., GLD-2) on an appropriate test mRNA (e.g., FIG. 4). In addition, the desired interacting proteins, when tethered in vivo, may cause polyadenylation by recruiting the endogenous cellular catalytic PAP subunit. (See U.S. Pat. Nos. 5,985,575 and 6,303,311 for descriptions of an appropriate tethered function assay.)

### B. Preparation of rPAP

In one embodiment, the present invention is a preparation of an rPAP or a preparation of a catalytic subunit of an rPAP. By “preparation” we mean a quantity of polymerase or subunit that has been separated from its natural environment. We mean to include both pure and crude preparations.

In another embodiment, the present invention is an isolated polynucleotide encoding an rPAP or catalytic subunit of the present invention.

The Examples below describe the preparation of GLD-2 and GLD-2 homologs. One of skill in the art would be able to identify other homologs by methods described below.

We also mean to include preparations where the catalytic subunit is in a complex with an RNA binding protein, whether or not the subunit is its native binding partner. For example, we specifically envision that one would wish to use the human catalytic subunit that we describe below in various combinations, such as the catalytic subunit linked covalently to any RNA binding protein (e.g., MS2 coat protein) or to its naturally occurring protein partners.

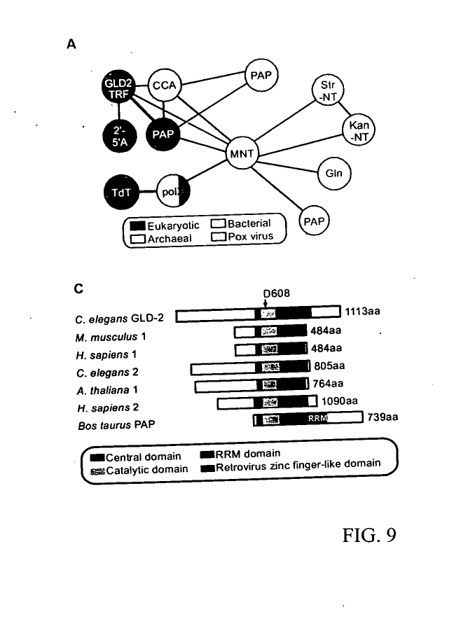

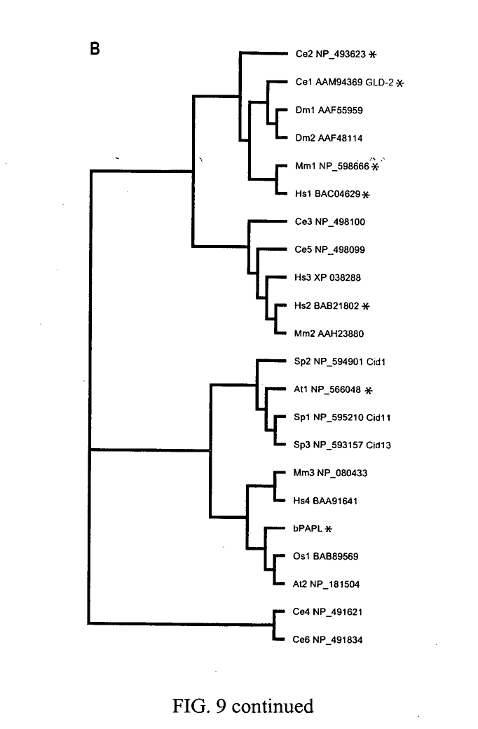

Most preferably, the rPAP or catalytic subunit is of derived from human rPAP1, as described below, or another human poly A polymerase subunit. One embodiment of the human enzyme is described at length in the Examples, most particularly FIG. 9. We demonstrate a sequence analysis that selects particular sequences as being homologs of the C. elegans GLD-2 sequence. Most particularly, we found that the H. sapiens sequence hRPAP1 (BAC04629) was an active GLD-2 homolog, as well as the mouse sequence mRPAP (NP_{—}598666) and yeast sequence TRF-5. (Applicants note that hRPAP1 is alternatively referred to as Hs-1 in this text. mRPAP is alternatively referred to as Mm-1.)

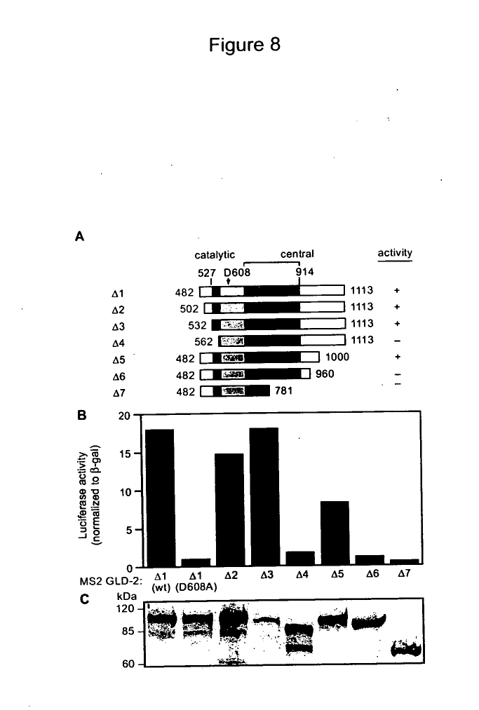

We refer both to the complete protein sequences as well as to fragments that possess catalytic activity. For example, we have detected polyadenylation activity by the enzyme upon tethering to RNA using either the complete protein sequence, or using portions of the protein corresponding to the predicted catalytic portion of the molecule. Portions of GLD-2 corresponding to amino acids 482-113, 502-1113, 532-1113 and 482-1000 all possess activity (FIG. 8).

Catalytic subunits and rPAPs of the present invention could be obtained in the following ways:

(1) By expression in bacteria, baculovirus or other expression systems, using purification methods familiar to those skilled in the art;

(2) By expression in Xenopus oocytes and other cells after introducing mRNA or DNA encoding proteins, using methods familiar to those skilled in the art; and/or

(3) By purification of the endogenous protein from unmanipulated human cells.

One of skill in the art could obtain proteins or peptides encoded by the sequences described herein in a number of ways. One typical way of obtaining an isolated polynucleotide encoding an rPAP or the catalytic subunit of an rPAP would be to design probes from the published sequences for GLD-2 and GLD-3 and isolate sequences encoding GLD-2 and GLD-3 from C. elegans or other organism of interest. For example, one might use the sequence of hRPAP-1 (found at GenBank BAC04629) to design probes to isolate this sequence from a human cell line. One would then wish to examine the sequence and protein products expressed from the sequence to determine what coding region is not necessary for catalytic activity.

Applicants note that the Examples below disclose that portions of the GLD-2 subunit may be eliminated with retention of catalytic activity. Therefore, by “catalytic subunit” we mean to include proteins or peptides which have non-essential portions deleted or non-essential or innocuous portions added. For example, one may modify the GLD-2 peptide as described below or above and still obtain a catalytically active peptide. We refer to these as “mutants retaining catalytic activity.”

### C. Ability to Identify rPAPs.

Of many predicted sequences related to rPAPs, we have identified some that actually are rPAPs and others that are not. In particular, the present invention is an rPAP and/or catalytic subunits from species, including, but not confined to Xenopus, S. cerevisae, C. elegans, S. pombe and human cells.

In one embodiment of the present invention, one would first align a candidate DNA sequence with one of the sequences presented in FIG. 9 and determine whether the candidate sequence has the catalytic domain and central domain corresponding to C. elegans GLD-2. The candidate sequence must possess the three catalytic carboxylates, and the residues required for positioning the nucleotide. These residues must be in the order and with the approximate spacing as that presented in FIG. 11.

Preferably, one would then wish to do an activity assay, most typically as described below in the tethered function analysis described in the Examples, to show that the sequence actually had PAP activity.

### D. Drug Discovery and Development.

rPAPs likely regulate multiple mRNAs. The rPAPs therefore have a diversity of biological functions, several of which provide attractive clinical targets for intervention with small molecules. Compounds that enhance or inhibit the activity of the rPAPs may perturb stem cell division, tumor cell growth or learning and memory, by disrupting regulation of key mRNAs.

Thus, molecules that affect the activity of a human rPAP may:

(1) affect differentiation of stem cells,

(2) affect division of stem cells,

(3) affect differentiation of cells in the hematopoeitic lineage,

(4) affect division of stem cells in the hematopoeitoic lineage,

(5) serve as cancer chemotherapies based on suppression of division of blood cells, and/or

(6) serve as cancer chemotherapies [or adjunct co-therapies] based on suppression of division in cells in other lineages.

The role of polyadenylation in learning and memory suggests that the same class of chemical compounds may be able to modulate these, and other, higher cortical processes. Since the enzyme may be required for the development of both eggs and sperm, inhibitors may reversibly prevent male of female fertility.

Therefore, one would wish to know whether a particular compound either enhanced or inhibited the activity of rPAP. One would most typically evaluate a compound by exposing the compound to a known rPAP, such as the GLD-2/GLD-3 enzyme described below, and determining whether the compound enhanced or inhibited the PAP activity of the enzyme. Preferably, one would wish to use an enzyme of human origin.

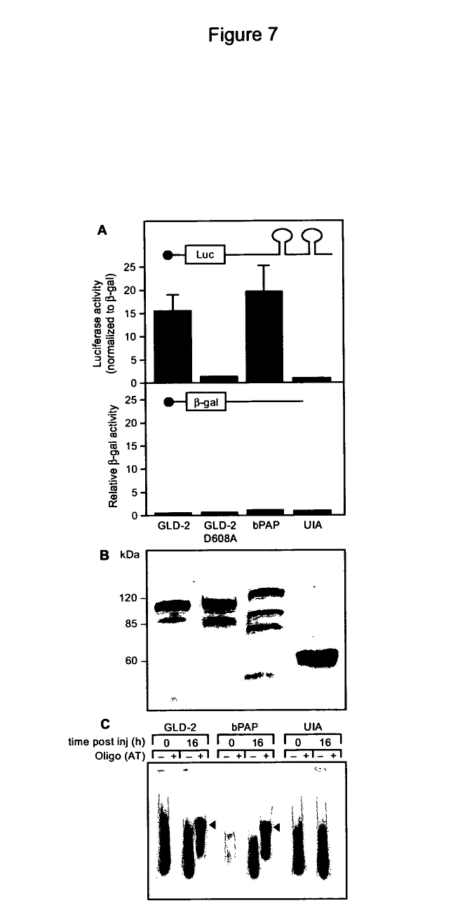

Polyadenylation activity would preferably be assessed using either an in vitro system or an in vivo expression system. In one embodiment in vitro, an mRNA encoding the protein is translated in vitro and assayed using an RNA added subsequently (as in FIG. 4C and 4D); in another in vitro embodiment, recombinant protein is used. In one in vivo embodiment, an mRNA or gene encoding the protein is introduced into cells. The activity is then assessed by monitoring either stimulation of translation, abundance or stability of the mRNA or its polyadenylation status (FIGS. 7, 8, 10).

In one embodiment, the RNA substrate is immobilized to a surface and the catalytic subunit or rPAP complex added. Tagged ATP is added, and the association of the tagged A with the RNA substrate monitored. For example, the incorporation of the tagged ATP onto the immobilized RNA can be monitored either by fluorescence anisotropy with appropriately conjugated ATP derivates, or by detecting incorporation of the tagged ATP onto the immobilized RNA.

In a second embodiment, the protein, RNA and tagged ATP are mixed in solution and the incorporation of the tagged ATP monitored as above.

Screening for molecules that enhance or inhibit these activities will follow practices commonly known among those skilled in the art.

Among the molecules so identified, secondary screens will be used to identify those that: specifically affect the desired PAP, and not the canonical nuclear PAP. To do so, the same assay will be performed, but using the canonical PAP in place of the rPAP or its catalytic subunit. Additional screens will identify those small molecules that exert their effects at the lowest concentration, established by titration, and subsequently identify those that are most efficacious in vivo. For this purpose, cell-based assays using tethered PAP provide one preferred embodiment. For example, inhibitors will prevent polyadenylation of mRNAs to which the PAP is tethered.

We believe rPAPs may be intimately involved in the following metabolic situations and thus, the determination of whether a compound enhances or inhibits PAP activity may be of extreme interest in the following situations:

i. Chemotherapy.

We have recently shown that TRF-5 of S. cerevisiae is a catalytic PAP, by tethering it to an mRNA in Xenopus oocytes. TRF-4 is a closely related protein with which TRF-5 interacts genetically, and may also possess PAP activity. Cells lacking TRF-4 are dramatically more sensitive to killing by cancer chemotherapeutic agents. This hypersensitivity is suppressed by overexpression of the PAP. Moreover, TRF-4 and TRF-5 genes are synthetically lethal in yeast, suggesting they collaborate in the same process.

The roles of TRF-4 and TRF-5 in cell death caused by cancer chemotherapeutic agents suggest new routes to develop chemotherapeutic agents. The mechanism of killing by these agents is the same. The mechanism of killing by these agents is the same in yeast and in human tumor cells (van der Zee, A. G., et al., Cancer Res. 51:5915-5920, 1991; Pommier, Y., et al., Drug Res. Updates 2:307-318, 1999; Carmichael, J. and Ozols, R. F., Exp. Op. Invest. Drugs 6:593-608, 1997). Thus anti-rPAP drugs are attractive for chemotherapy.

ii. Stem Cell Regulation.

rPAPs control the decision between division and differentiation in C. elegans stem cells (Wickens, M., et al., Translational Control of Gene Expression, eds. Hershey, J. W. B., et al., Cold Spring Harbor Laboratory Press, Cold Spring Harbor, N.Y., pp. 295-370, 2000; Zhao, J., et al., Micro. Mol. Biol. Rev. 63:405-445, 1999; Bienroth, S., et al., EMBO J. 12:585-594,1993). Thus drugs or genes that influence its activity likely would perturb that balance, or perhaps shift pathways of differentiation. From our work in C. elegans, we predict that anti-rPAP compounds would enhance proliferation of stem cells (Wickens, M., et al., supra, 2000).

iii. The Brain.

Cytoplasmic polyadenylation is critical for learning and memory (Patil, C. and Walter, P., Curr. Opin. Cell Biol. 13:349-355, 2001; Jacobson, A., Translational Control, eds. Hershey, J. W., et al., Cold Spring Harbor Laboratory Press, Plainview, N.Y., pp. 451-480,1996; Jacobson, A. and Peltz, S. W., Ann. Rev. Biochem. 65:693-739,1996; Dickson, K. S., et al., Mol. Cell. Biol. 19:5707-5717, 1999). However, the poly(A) adding enzyme responsible is not yet known. We suspect that the enzyme is in fact the rPAP we have identified. It already is clear that genetic lesions that disrupt cytoplasmic polyadenylation enhance learning and memory (Richter, J. personal communication). Thus the poly(A) polymerase involved is a particularly attractive clinical target.

### EXAMPLES

(Applicants note that hRPAP1 is alternatively referred to as Hs-1 in this text. mRPAP is alternatively referred to as Mm-1.)

### A. Regulatory Cytoplasmic Poly(A) Polymerase in C. elegans

Results and Discussion

Here we disclose that the Caenorhabditis elegans gene gld-2, a regulator of mitosis/meiosis decision and other germine events (Kadyk, L. C. and Kimble, J., Development 125:1803-1813, 1998), encodes the catalytic moiety of a cytoplasmic poly(A) polymerase (PAP) that is associated with P granules in early embryos.

Importantly, the GLD-2 protein sequence has diverged substantially from that of conventional eukaryotic PAPs and lacks a recognizable RRM (RNA recognition motif)-like domain. GLD-2 has little PAP activity on its own, but is stimulated in vitro by GLD-3. GLD-3 is also a developmental regulator, and belongs to the Bicaudal-C family of RNA binding proteins (Eckmann, C., et al., Dev. Cell 3:697-710, 2002). We suggest that GLD-2 is the prototype for a class of regulatory cytoplasmic PAPs that are recruited to specific mRNAs by a binding partner, thereby targeting those mRNAs for polyadenylation and increased expression.

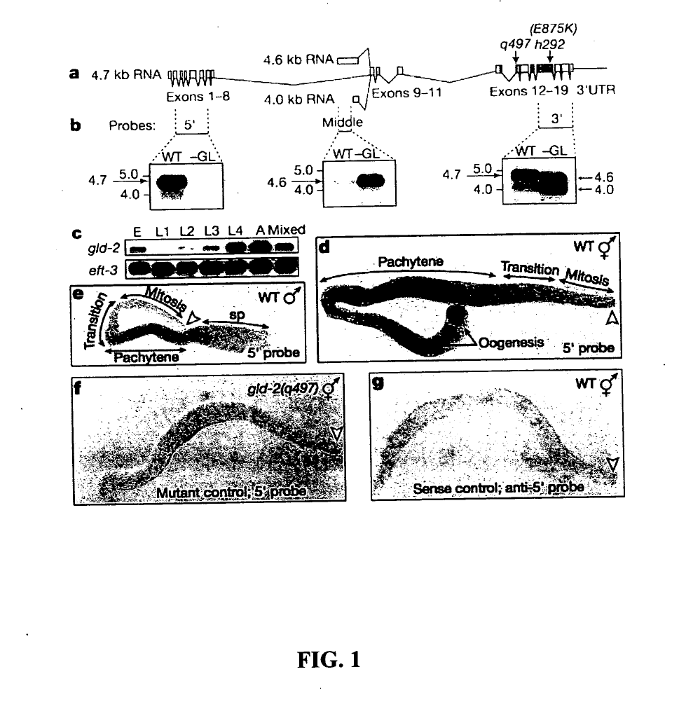

As disclosed below, we cloned the gld-2 gene and analysed its transcripts (FIG. 1). The gld-2 genomic region was identified by mutant rescue and RNA-aided interference (RNAi; see Methods) as well as elucidation of the molecular lesions in two gld-2 mutants (see below). The gld-2 gene encodes multiple mRNAs (FIG. 1a, b). A 5′ probe detected a 4.7-kilobase (kb) band in wild-type poly(A)⁺ RNAs, but not in RNA from germline-less mutants (FIG. 1b, left). Therefore, this 4.7-kb mRNA appears to be germline-specific. Middle and 3′ probes detected two somatic gld-2 RNAs, of 4.6 and 4.0 kb (FIG. 1b, middle and right). These smaller gld-2 mRNAs harbour distinct 5′ terminal exons spliced to common exons (FIG. 1a). Two gld-2 mutations identified genetically (Kadyk, L. C. and Kimble, J., et al., supra, 1998) carried lesions in common exons: a predicted null mutant, gld-2(q497), is a premature nonsense codon, and gld-2(h292) is a missense mutation (E875K) (FIG. 1a).

Because of our interest in gld-2 germline functions, we focused on its 4.7-kb mRNA. Northern analysis (FIG. 1b, left) showed that this mRNA was abundant in embryos, fourth larval stages (L4s) and adults (FIG. 1c); in situ hybridization showed that it was abundant in the meiotic pachytene region and in oogenesis (FIG. 1d, e), but decreased during spermatogenesis (FIG. 1e). We did not detect the mRNA in putative null mutant gld-2(q497) (FIG. 1f), or with a sense-strand probe (anti-5′) (FIG. 1g). Therefore, gld-2 is expressed in the germ line and is developmentally regulated.

Database searches revealed that GLD-2 protein belongs to the DNA polymerase β-like superfamily of nucleotidyltransferases (NT) (FIG. 2a; Holm, L. and Sander, C., Trends Biochem. Sci. 20:345-347,1995; Aravind, L. and Koonin, E. V., Nucleic Acids Res. 27:1609-1618, 1999). Specifically, GLD-2 is a group 2 NT member, including DNA polymerase a of Saccharomyces cerevisiae (also known as pol κ and Trf4p) and eukaryotic PAPs (FIG. 2a). GLD-2 architecture and sequence is divergent from that of canonical PAPs (FIG. 2b, d), but similar to a different cluster of NT family members (FIG. 2e). GLD-2 contains three critical carboxylate side chains essential for catalytic activity (FIG. 2c, red) present in all DNA polymerase β superfamily members; furthermore, GLD-2 possesses putative ATP-interacting residues (FIG. 2c, green; FIG. 2d, green). Classical PAPs have a catalytic region (FIG. 2c, gold), a ‘central’ domain (FIG. 2c, blue), and an RRM-like region (FIG. 2c, violet) (Martin, G., et al., EMBO J. 19:41934203, 2000; Bard, J., et al., Science 289:1346-1349, 2000). By sequence comparison, GLD-2 harbours catalytic and central domains (FIG. 2b, d, colour-coded overlines), but is highly diverged from classical eukaryotic PAPs, including C. elegans PAP-1 (C. Luitjens and M. W., unpublished results) (FIG. 2d). Classical PAPs show extensive amino-acid conservation among themselves, but limited conservation with GLD-2 (FIG. 2d, black and grey boxes). Outside its catalytic and central domains, GLD-2 shares little similarity to canonical PAPs; in particular, GLD-2 has no apparent RRM-like region (FIG. 2b), which is thought to be critical for PAP RNA binding (Martin, G., et al., supra, 2000; Bard, J., et al., supra, 2000). Therefore, GLD-2 shares some key features with classical PAPs, but is divergent in motif architecture and amino acid sequence.

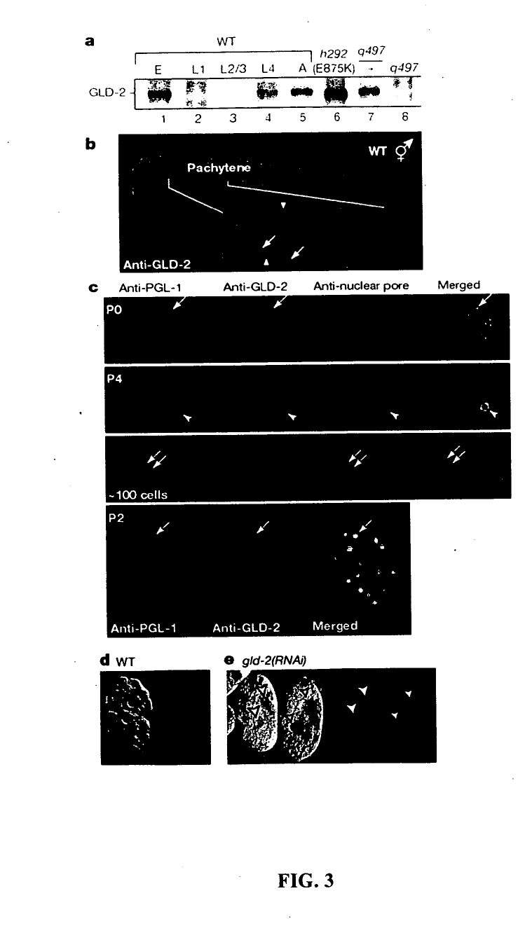

To examine GLD-2 protein, we generated polyclonal antibodies to the amino-terminal region (FIG. 2b) and detected a prominent protein of relative molecular mass 125,000 (Mᵣ 125K) on western blots (FIG. 3a, lanes 1, 4, 5). This protein, which corresponds in size to the predicted product of the germline gld-2 mRNA, was detected in gld-2(h292) homozygotes and gld-2(q497)/+heterozygotes (FIG. 3a, lanes 6, 7), but not in gld-2(q497) homozygotes (FIG. 3a, lane 8). Pre-immune serum did not recognize this band, but detected others that served as a loading control (not shown). We conclude that the α-GLD-2 antibody recognizes GLD-2, that the gld-2(h292) mutant produces a nearly wild-type level of protein and that gld-2(q497) is a strong loss-of-function or null allele.

By immunocytochemistry, GLD-2 was found to be predominantly cytoplasmic in both germ line (FIG. 3b) and early embryo (FIG. 3c). Within the germ line, GLD-2 was detectable in the mitotic region and became abundant during pachytene and oogenesis (FIG. 3b). GLD-2 decreased during spermatogenesis in both sexes, and was undetectable in mature sperm (not shown). In early embryos, GLD-2 was diffuse in the cytoplasm of early P0 embryos, co-localized with P granules in late P0 embryos and remained associated with P granules in germline blastomeres (FIG. 3c, not shown). P granules are essential for germline development (Seydoux, G. and Strome, S., supra, 1999; Kawasaki, I., et al., Cell 94:635-645,1998). In ˜100-cell embryos, GLD-2 was undetectable.

Given its presence in oocytes and early embryos, we tested whether GLD-2 was required for embryogenesis. To deplete both maternal and zygotic gld-2 mRNAs, wild-type adult hermaphrodites were treated with double-stranded RNA corresponding to either the gld-2 germline-specific region (exons 2-8) or its common region (exons 16-18) to produce gld-2(RNAi) embryos (see Methods). In both cases, most gld-2(RNAi) embryos failed to hatch (99%, n>500 in 26-36 hour period after treatment). To visualize chromosomes in gld-2(RNAi) embryos, we used a strain carrying a histone::GFP transgene (AZ212) (Praitis, V., et al., Genetics 157:1217-1226, 2001). Whereas mock-treated AZ212 embryos cleaved normally (FIG. 3c), gld-2(RNAi) AZ212 embryos did not cleave and possessed malformed nuclei in clusters (FIG. 3e). We conclude that gld-2 activity is required for embryogenesis, and that GLD-2 protein co-localizes with P granules.

A specific interaction between GLD-2 and another germline regulator, GLD-3 (Eckmann, C., et al., supra, 2002), was discovered in yeast two-hybrid screens. Specifically, using GLD-2 as ‘bait’, 2,000,000 transformants were screened and 30 gld-3 cDNAs (T07F8.3) found; using GLD-3 as bait, 1,500,000 transformants were screened and 94 gld-2 cDNAs recovered. To identify the region of GLD-2 critical for GLD-3 binding, GLD-2 variants were assayed for GLD-3 interaction. A GLD-2 fragment comprising both catalytic and central domains was essential (amino acids 544-924) (FIG. 4a). A GLD-2-E875K mutant, designed after gld-2(h292) (Kadyk, L. C. and Kimble J., supra, 1998), interacted poorly with GLD-3 (FIG. 4a, E875K and Δ7). Indeed, β-galactosidase activity was reduced 7- to 16-fold by GLD-2(h292)-E875K (FIG. 4a, compare for example Δ2 to Δ7), but GLD-2 levels were equivalent (FIG. 4b). Importantly, GLD-2-E875K was present at normal levels in C. elegans (FIG. 3a, lane 6), even though it disrupts gld-2 function. We conclude that GLD-2 binds specifically to GLD-3, and that GLD-2-E875K is defective in GLD-3 binding. Therefore, the GLD-2/GLD-3 interaction appears to be important for development.

Given its sequence similarity to nucleotidyltransferases and its cytoplasmic location, we considered that GLD-2 might be a cytoplasmic PAP, even though its architecture and sequence diverged substantially from classical PAPs. To test this idea, we initially assayed incorporation of radiolabelled ATP into an RNA substrate. Specifically, GLD-2 was translated in vitro, either on its own or together with GLD-3. The in vitro translation mixture was incubated with ³²P-ATP and an unlabelled poly(A) substrate, and incorporation of label into acid-insoluble material was measured (see Methods). GLD-2 on its own had low but readily detectable activity, as do the S. pombe enzymes (Saitoh, S., et al., supra, 2002). We also measured incorporation in three control reactions (no protein and two GLD-2 mutants together with GLD-3). GLD-2-D608A was designed to abolish the catalytic site (FIG. 2c) and GLD-2-E875K was used to disrupt GLD-3 binding (FIG. 4a). The control reactions yielded no measurable ³²P-ATP incorporation (FIG. 4c). From these experiments, we argue that GLD-2 is in fact a nucleotidyltransferase and that both its predicted active site and GLD-3 binding region are essential for enzymatic activity.

We next analyzed the products of the GLD-2/GLD-3 nucleotidyltransferase activity by electrophoresis and autoradiography (FIG. 4d). To this end, reactions were done as described above, except that C₃₅A₁₀ (see Methods) was used as substrate. Two exposures of the same autoradiogram are shown (FIG. 4c). As a marker, C₃₅A₁₀ was 3′ end-labelled with cordycepin triphosphate ([α-³²P] 3′ dATP) (C₃₅A₁₀*dA; FIG. 4d left, lane 1). GLD-2 by itself exhibited modest incorporation from ATP into bands that extended the substrate by only one or a few nucleotides (FIG. 4d left, lane 2). In contrast, GLD-2 plus GLD-3 stimulated incorporation, resulting in more product with a ‘ladder’ of poly(A) extending the substrate more than 30 adenosines (FIG. 4d, lane 4). The ladder mimics the activity of bovine nuclear PAP (bPAP), but is less efficient (FIG. 4d, compare lanes 4 and 7). This difference may reflect the fact that bovine PAP acts as a monomer, whereas GLD-2 PAP activity is dependent on the interaction of two dilute proteins. Furthermore, although abundant products had only two or three nucleotides added (asterisks in FIG. 4d, lane 4), more-minor products had as many as 70 additional nucleotides. We conclude that GLD-2/GLD-3 can catalyse the addition of a poly(A) tail to an RNA substrate.

Four controls support the conclusion that GLD-2 is a PAP. First, GLD-2 PAP activity was abolished by a site-directed mutation in the inferred active site (D608A) (FIG. 4d, lane 5). Importantly, GLD-2-D608A level is equivalent to that of wild-type GLD-2 in the same assay (FIG. 4d SDS-polyacrylamide gel electrophoresis, SDS-PAGE, compare lanes 4 and 5). Thus, the GLD-2 putative active site is required for AMP addition in vitro. Second, GLD-2 PAP activity was abolished by the E875K mutation (FIG. 4d, lane 6), which disrupts GLD-2/GLD-3 binding (FIG. 4a). The GLD-2-E875K level was equivalent to wild-type GLD-2 (FIG. 4d, compare lanes 4 and 6). Third, GLD-2-dependent incorporation is substrate dependent and requires ATP (not shown). Thus, replacement of ATP with GTP, CTP or UTP did not yield incorporation onto the substrate. Finally, products produced by GLD-2 plus GLD-3 were selectively retained on oligo(dT) cellulose, suggesting they were polyadenylated (not shown).

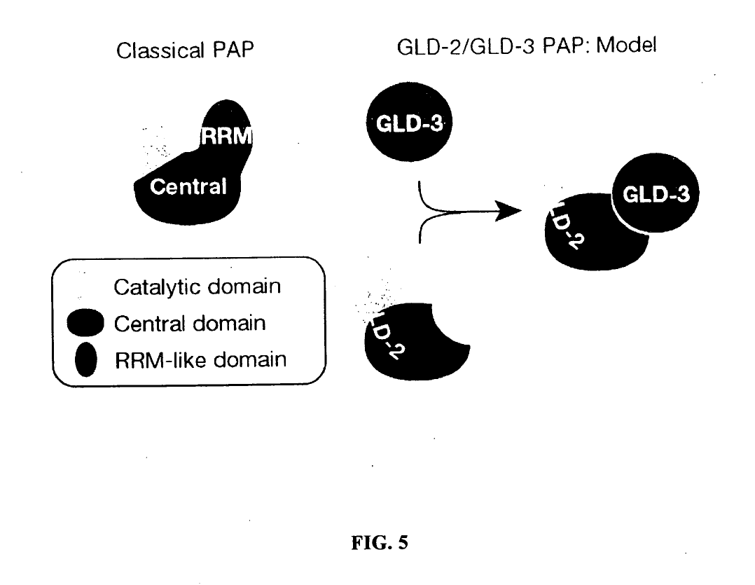

The GLD-2/GLD-3 enzyme represents a new type of poly(A) polymerase (FIG. 5). Canonical PAPs, which include nuclear and cytoplasmic enzymes, are all closely related (Colgan, D. F. and Manley, J. L., Genes Dev. 11:2755-2766,1997; Kashiwabara, S.-i., etal., Dev. Biol. 228:106-115, 2000; Kyriakopoulou, C. B., et al., J. Biol. Chem. 276, 33504-33511, 2001; Topalian, S. L., et al., Mol. Cell. Biol. 21:5614-5623, 2001); they are monomeric and possess three key domains (FIG. 5, left) (Martin, G., et al., supra, 2000; Bard, J., et al., supra, 2000). By contrast, GLD-2 appears to function as a heterodimer (FIG. 5, right). GLD-2 harbours the catalytic and central domains; GLD-3 has five consecutive K homology (KH)-related motifs (Eckmann, C., et al., supra, 2002) which may, at least in part, substitute for the RRM domain of classical PAPs. In the simplest view, GLD-2 and GLD-3 act together as a heterodimer to accomplish what classical PAPs do on their own. However, we suggest that GLD-2 is tailored for a more regulatory role than that typical of classical PAPs. For example, GLD-3 is likely to provide sequence specificity to the GLD-2 catalytic activity, and GLD-2 may interact with additional partners to expand its repertoire of regulation.

GLD-2 and GLD-3 are likely to function together during nematode development. First, GLD-2 and GLD-3 have similar, albeit not identical, functions in germline development and embryogenesis (Kadyk, L. C. and Kimble, J., supra, 1998; Eckmann, C., et al., supra, present disclosure). Second, both are cytoplasmic and associated with P granules (Kadyk, L. C. and Kimble, J., supra, 1998, present disclosure), large complexes of RNA and protein that are critical for germline development (Seydoux, G. ad Strome, S., supra, 1999; Kawasaki, I., et al., supra, 1998). GLD-2 and GLD-3 may polyadenylate mRNAs associated with P granules (for example, nos-2; Subramaniam, K. and Seydoux, G., Development 126:4861-4871, 1999) or may be stored there for segregation to germline blastomeres. GLD-2 may be targeted to specific mRNAs by GLD-3, which is a Bic-C family KH protein. Other KH proteins (FMRP, NOVA, hnRNPK) bind RNAs through sequence-specific interactions (Jensen, K. B., et al., Proc. Natl. Acad. Sci. USA 97:5740-5745, 2000; Brown, V., et al., Cell 107:477-487, 2001; Darnell, J. C., et al., Cell 107:4890-499, 2001; Ostrareck, D. H., et al., Cell 89:597-606, 1997). GLD-2 may also be targeted to specific mRNAs indirectly via the interaction of GLD-3 with FBF. FBF is a sequence-specific RNA-binding protein and member of the PUF family (Wickens, M., et al., Trends Genet. 18:150-157, 2002). PUF proteins appear to repress mRNAs by promoting poly(A) removal (Wickens, M., et al., supra, 2002). GLD-3 antagonizes FBF, and works with GLD-2 to promote poly(A) addition (this work). Therefore, GLD-2/GLD-3 may switch FBF from a repressive to an activating mode.

Regulatory cytoplasmic PAPs of the GLD-2/GLD-3 class may be common. Within the large superfamily of DNA polymerase β-like nucleotidyltransferases, several are closely related to GLD-2 (FIG. 2e). To date, most have no assigned function, but Schizosaccharomyces pombe Cid13 and Cid1 appear to be rcPAPs (Saitoh, S., et al., Cell 109:563-573, 2002). The similarity between GLD-2 and Cid1 is particularly striking, as both are involved in cell cycle control. GLD-2 promotes entry into meiosis at the expense of mitosis (Kadyk, L. C. and Kimble, J., supra, 1998), and Cid1 inhibits mitosis (Wang, S. W., et al., Mol. Cell. Biol. 20:3234-3244, 2000). We suggest that GLD-2 and Cid1 may in fact be components of an ancient regulatory circuit controlling the cell cycle, and that other GLD-2 relatives may similarly be regulatory cytoplasmic PAPs.

### Methods

Molecular cloning of gld-2. Three-factor mapping places gld-2 0.05 map unit to the right of bli-4. Cosmids in this region were injected into strain JK1716 [bli-4(e937) gld-2(q497)/dpy-5(e61) unc-13(e51)] or strain JK1732 [bli-4(e397) gld-2(h292)/dpy-5(e61) unc-13(e51)]. Cosmid ZC308 gave ˜4% germline rescue.

Transcript analyses. Northern blots were performed as described (Sambrook, J., et al., Molecular Cloning: A Laboratory Manual (Cold Spring Harbor Laboratory Press, New York, N.Y., 1989). Templates for making RNA probes (gld-2 5′, middle, 3′; eft-3) were made by polymerase chain reactions (PCRs) from pJK830, pJK831, pJK832 and pBluescript-eft-3 (gift from P. Anderson). To determine the gld-2 3′ end, semi-nested PCR was performed using AAE.1, a C. elegans mixed-stage oligo(dT) primed complementary DNA library (gift from A. Puoti). One PCR product was confirmed and sequenced. A stretch of 22 As was found at the end of the 3′ untranslated region (UTR). To determine the gld-2 5′ ends, reverse transcriptions (RT) were performed using SuperScript II Reverse Transcriptase (Gibco BRL) and poly(A)⁺ RNA from either wild-type mixed-stage worms or gip-1(q224) mutants raised at 25° C., which have no germ line. The resultant cDNAs were then used as templates for semi-nested PCR with SL1 (a trans-spliced leader in C. elegans) as the constant 5′ primer. All PCR products were cloned into pSTBlue-1 and sequenced. The 4.7-kb mRNA is SL1 trans-spliced, comprises 19 exons including an 86-nucleotide 5′ UTR and 1,105-nucleotide 3′ UTR.

Antibody production, western blot and immunocytochemistry. Polyclonal antibodies were generated from rabbits using a keyhole limpet haemocyanin (KLH)-conjugated peptide corresponding to GLD-2 amino acids 108-127 (Genemed Synthesis) or from rats using a GST-GLD-2 fusion protein carrying amino acids 13-330 of GLD-2. Rabbit anti-PGL-1 antibody was a gift from S. Strome. Monoclonal antibody 414, the anti-nuclear pore monoclonal, was purchased from BABCO. Western blots were performed using the GLD-2 peptide antibody as described (Sambrook, J., et al., supra, 1989). Immunocytochemistry followed published procedures (Chrittenden, S. L., and Kimble, J., Cell: A Laboratory Manual, pp.108.1-108.9, Cold Spring Harbor Laboratory Press, Cold Spring Harbor, N.Y., 1998) using the GST-GLD-2 fusion-protein antibody, which was specific for GLD-2 as demonstrated on gld-2(q497) extruded germ lines and gld-2(RNAi) embryos.

RNAi. Double-stranded RNAs (dsRNAs) were made using gld-2 cDNAs (pJK830, exons 2-8 or pJK831, exons 16-18) as templates. Young adults were either injected with 2 μg μl⁻¹ gld-2 dsRNA or soaked in 10 μl of 2 μg μl⁻¹ gld-2 dsRNA for 12 hours at 20° C. or mock-treated by injection with M9 buffer. Embryos were collected at defined intervals after treatment and processed together.

Poly(A) polymerase assay. Proteins were in vitro translated using the TNT coupled transcription-translation system (Promega), and assayed using buffer conditions essentially as described (Lingner, J., et al., J. Biol. Chem. 266:8741-8746, 1991). For scintillation counting, poly(A) (Roche) was used as substrate. For gel assays, we used RNA oligo, C₃₅A₁₀ (Dharmacon), a 45-nucleotide and supplemental 1 mM MgCl₂. Products were analyzed on 12% sequencing gels.

### B. Novel Human Poly(A) Polymerases

In this Example, we first tested the hypothesis that GLD-2 is the catalytic subunit of a novel, heterodimeric PAP. We determined whether recruitment of an rPAP to an mRNA is sufficient to stimulate PAP activity. To do so, we tethered GLD-2 to an mRNA in Xenopus oocytes using a foreign RNA-binding protein, MS2 coat protein. The chimeric protein added poly(A) selectively to those mRNAs with MS2 binding sites, and stimulated their translation. This assay also enabled the identification of new rPAPs without knowledge of their protein partners or mRNA targets. Indeed, we identified novel human and mouse proteins that possess polyadenylation activity, and so are putative new members of the family of regulatory PAPs.

Methods

DNA Constructs

p3HA-MSP. Three HA tags were inserted into pET15b-CPEB to replace the His-tag (p3HA-CPEB, Dickson, unpublished data). MS2 fragment of pET-MS2 (Gray, N. K., et al., EMBO J. 19:47234733, 2000) was ligated into p3HA-CPEB from Ndel-Nhel. This vector contains the T7 promoter, followed by three HA tags, and two tandem copies of the MS2 coat protein (p3HA-MSP).

MS2 fusions. pMS2-U1A and pMS2-bPAP were previously described (Dickson, K. S., et al., J. Biol. Chem. 276:41810-41816, 2001). GLD-2 and truncated GLD-2 mutants were PCR amplified from pLW48. Hs1 and Hs2_were PCR amplified from reverse transcribed DNA derived from a human breast cancer (MDA231) cell. Mm1 was PCR amplified from reverse-transcribed DNA derived from a mouse macrophage RAW264.7 cell. At1 was PCR amplified from H2D8 plasmid (Arabidopsis biological resource center). Ce5 was PCR amplified from reverse transcribed DNA derived from mixed stage C. elegans. p3HA-MSP was cut either with Nhel and Xhol or with Nhel and Clal to remove CPEB. All the PCR products except Ce5 were ligated into p3HA-MSP from Nhel-Xhol. Ce5 PCR product was ligated into p3HA-MPS from Nhel-Clal. The active site mutant of GLD-2 (GLD-2 D608A) was created from p3HA-GLD-2 using QuikChange mutagenesis system according to the manufacturer instruction (Stratagene). Thus each plasmid carrying a fusion with MS2 coat protein contains the T7 promoter, followed by three HA tags, two tandem copies of MS2 coat protein, and the protein to be tested. Plasmids were linearized with HindIII, except with Mm1, Hs1 and Ce5 (which are cleaved with Cla1) and At1 (cleaved with Ssp1).

mRNA reporterplasmids. pLG-MS2 (luciferase) and pJK350 (β-galactosodase) plasmids have been described (Dickson, K. S., et al., supra, 2001). pLG-MS2 was linearized with Bg/II and transcribed with T7 RNA polymerase. pJK350 was linearized with HindIII and transcribed with SP6 RNA polymerase.

In vitro transcription. Plasmids were linearized with restriction enzymes as indicated above, and then transcribed with T7 or SP6 RNA polymerase. Transcriptions were performed in the presence of 6 mM 7meGpppG.

RNA isolation and poly(A) selection. To prepare labeled RNA for injection, luciferase mRNA was transcribed in vitro using T7 RNA polymerase in the presence of [α-³²P]UTP, as described (Dickson, K. S., et al., supra, 2001). After injection into oocytes, RNA was extracted using TRI reagent according to the manufacturer instructions (Sigma). The extracted RNA was further purified using RNeasy Mini Kit (Qiagen). Poly(A)⁺ mRNA was separated from poly(A)⁻ RNA by the PolyATract mRNA isolation system, using biotinylated oligo(dT), according to the manufacturer instructions (Promega). RNA was analyzed by electrophoresis through a denaturing formaldehyde-agarose gel followed by autoradiography.

Assays of fusion proteins using Xenopus oocytes. Oocyte injections and manipulations were performed essentially as described (Gray, N. K., et al., supra, 2001; Dickson, K. S., et al., supra, 2001). Stage VI Xenopus oocytes were injected first with the mRNA encoding the fusion protein. 6 hours later, they were injected with a mixture of luciferase and β-galactosidase mRNA, with and without MS2 binding sites respectively. Luciferase and β-galactosidase enzyme assays were performed as described (Gray, N. K., et al., supra, 2001).

Identification of GLD-2 relatives and sequence alignments. With C. elegans GLD-2 protein sequence as the query sequence (Wang, L., et al., supra, 2002), we ran the BlastP program using the nr (All non-redundant GenBank CDS translations+RefSeq Proteins+PDB+SwissProt+PIR+PRF) database at the NCBI BLAST server. We selected all “hits” with a cut-off e-value of 1e-10 for the analyses presented in FIG. 9, with the exception of two Anopheles gambiae sequences (EAA01442 and EAA09603), two Cryptococcus neoformans sequences (AAN75161 and AAN75620), one Plasmodium yoelii sequence (EM16601), one Giardia lamblia sequence (EM16601), two Plasmodium falciparum sequences (NP_{—}701679 and NP_{—}700626), and one Neurospora crassa sequence (EAA26901). We aligned the 20 sequences most highly related to GLD-2 (together with C. elegans GLD-2 and bovine poly(A) polymerase) using the “Superfamily” program. Based on the multiple sequence alignment, an unrooted tree was created with a phylogenetic tree program (http://www.ebi.ac.uk/clustalw) using neighbor joining method (Saitou, N. and Nei, M., Mol. Biol. Evol. 4:406-425,1987), setting on Kimura correction of distances (Kimura, M., The Neutral Theory of Molecular Evolution, Cambridge University Press, Cambridge, 1983).

Results

Tethered GLD-2 is an Efficient Poly(A) Polymerase

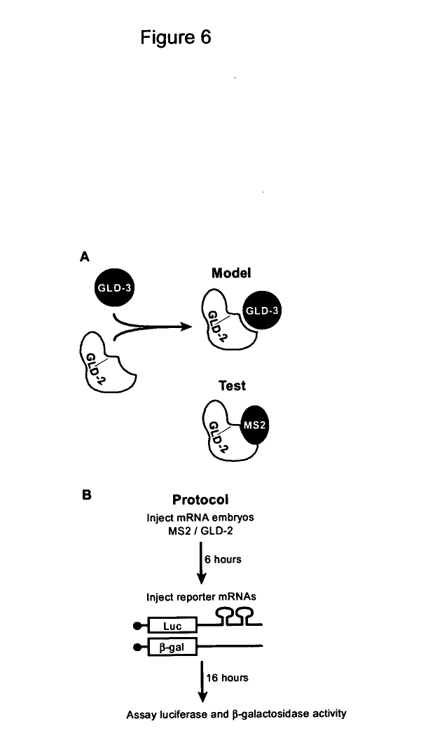

GLD-2's polyadenylation activity is stimulated by its interaction with a putative RNA-binding protein, GLD-3 (Wang, L., et al., supra, 2002). We proposed that the GLD-2/GLD-3 complex is the active form in vivo (FIG. 6A, “Model”). If GLD-3 stimulates activity by bringing the PAP to its substrate, then bringing GLD-2 there by other means also should stimulate its polyadenylation activity. To test this idea, we tethered GLD-2 to a reporter mRNA using MS2 coat protein (FIG. 6A, Test; See U.S. Pat. No. 5, 985,575 and 6,313,311). We used Xenopus oocytes for this purpose, since they provide a robust assay for cytoplasmic polyadenylation and its effects in vivo (Gray, N. K., et al., supra, 2001; Dickson, K. S., et al., supra, 2001).

Xenopus oocytes were first injected with an mRNA encoding a fusion of MS2 coat protein and GLD-2 (MS2/GLD-2) or with mRNAs encoding control proteins (FIG. 6B). After allowing six hours for synthesis of the fusion protein, two reporter mRNAs were co-injected: a luciferase mRNA bearing MS2 binding sites in its 3′UTR, and a β-galactosidase mRNA lacking MS2 sites (FIG. 6B). Luciferase and β-galactosidase activities were measured 16 hours later.

MS2/GLD-2 stimulates expression of the luciferase mRNA roughly 16-fold (FIG. 7A). Stimulation is specific, since it requires the MS2 RNA binding sites: the activity of the β-galactosidase reporter, which lacks binding sites, is not enhanced (FIG. 7A). Furthermore, a point mutation in GLD-2 (D608A), designed to inactivate the catalytic center (Aravind, L. and Koonin, E. V., supra, 1999; Martin, G. and Keller, W., supra, 1996), abolished translational stimulation by the MS2/GLD-2 fusion protein (FIG. 7A). MS2/GLD-2 is comparable in activity to MS2/bPAP, a fusion between MS2 and bovine nuclear PAP (FIG. 7A, “bPAP,” Dickson, K. S., et al., supra, 2001). As expected, MS2/U1A did not stimulate translation whether or not sites were present (FIG. 7A). The level of each MS2 fusion protein in the oocytes was comparable, as assessed by Western blotting (FIG. 7B). We conclude that GLD-2 specifically stimulates translation when bound to an mRNA in oocytes.

MS2/GLD-2 protein catalyzed polyadenylation of the reporter to which it was tethered (FIG. 7C). Luciferase mRNAs from oocytes containing MS2/GLD-2 were efficiently retained by oligo(dT) sepharose (FIG. 7C). Virtually none of the mRNA was retained immediately after injection, but most of it was retained after the 16 hour incubation (FIG. 7C; GLD-2, 0 vs 16 h). MS2/GLD-2 and MS2/bPAP had comparable polyadenylation activities (FIG. 7C). MS2/U1A possessed no detectable polyadenylation activity, further corroborating specificity (FIG. 7C).

The Minimal Region Required for Activity

GLD-2 posses hallmark features of the β-nucleotidyl transferase family (Aravind, L. and Koonin, E. V., supra, 1999). These include three aspartate acid residues and several amino acids that bind the nucleotide (FIG. 8A). To identify the minimum contiguous portion of GLD-2 capable of supporting polyadenylation, we prepared N- and C-terminal deletions of GLD-2, each fused to MS2 coat protein. Of the 1134 amino acids of full-length GLD-2, 532 and 113 could be pared from the N and C temini respectively, without significantly reducing activity (FIG. 8). Further encroachments of only 30 amino acids at the N-terminus, or 40 at the C-terminus, caused substantial decreases in activity. Each mutant protein was present at a comparable level in the oocyte, including those without activity (FIG. 8C). We conclude that the minimum contiguous portion of GLD-2 needed for catalysis corresponds roughly to the catalytic and central domains, and contains the key residues conserved among β-nucleotidyl transferases.

New Mouse and Human PAPs

The members of the β-nucleotidyl transferase family include several different biochemical activities, including DNA polymerases, CCA-adding enzymes, and 2′-5′ oligo(A) synthetases, as well as canonical eukaryotic PAPs (FIG. 9A; Aravind, L. and Koonin, E. V., supra, 1999). Sequences more closely related to GLD-2 than to canonical PAPs are detected throughout eukaryotes. The twenty most closely related sequences are depicted in a dendrogram in FIG. 9B.

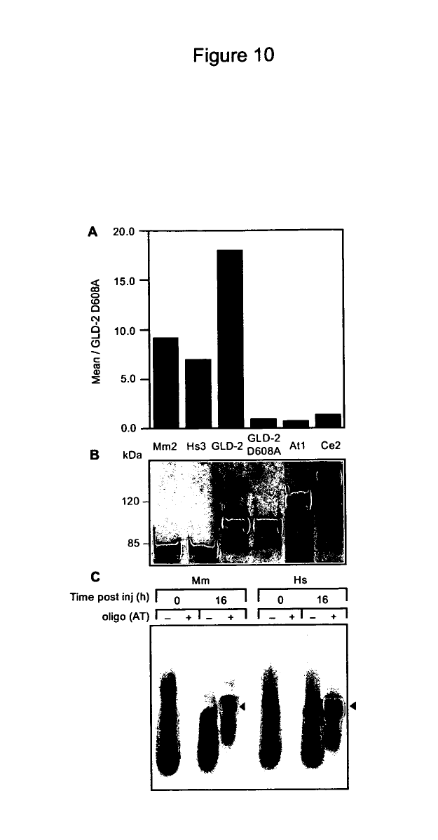

To elucidate which of these GLD-2 relatives were poly(A) polymerases, we tethered several candidates to a reporter mRNA using MS2 coat protein. We tested candidates from human, mouse, C. elegans, and Arabidopsis: each possesses the catalytic and central domains related to GLD-2 (FIG. 9C; FIG. 11). As shown in FIG. 10A, one human candidate (Hs1) and one mouse candidate (Mm1) were active. Their activities were approximately 50% that of GLD-2. The abundance in oocytes of the Hs1, Mm1 and GLD-2 fusion proteins was comparable (FIG. 10B). Hs1 and Mm1 both added poly(A) to the reporter mRNA, confirming that they possess poly(A) polymerase activity (FIG. 10C).

Several genes tested were inactive in the assay. These include candidates from Arabidopsis (At1), C. elegans (Ce5) and H. sapiens (Hs2) genomes (FIG. 10C). Since the proteins were produced in the oocyte, the simplest explanation is that only some, and not all, GLD-2 relatives are in fact poly(A) polymerases.

Discussion

Our findings support the model that GLD-2 gains activity by recruitment to its mRNA substrate (Wang, L., et al., supra, 2002). Tethering the protein to an mRNA results in polyadenylation and translational stimulation. In the simplest view, RNA binding partners of GLD-2 bring the protein to the 3′ end of the mRNA, providing sequence specificity.

Many enzymes that polymerize nucleotides into nucleic acid gain specificity by interaction with distinct partners. Replication and transcription enzymes follow this principle. In nuclear polyadenylation, the canonical PAP is brought to the pre-mRNA through interactions with a multisubunit RNA-binding protein, Cleavage and Polyadenylation Specificity Factor (CPSF; Minvielle-Sebastia, L. and Keller, W., supra, 1999; Colgan, D. F. and Manley, J. L., supra, 1997; Wahle, E. and Ruegsegger, U., supra, 1999). Cytoplasmic polyadenylation in Xenopus oocytes requires a distinct cytoplasmic form of CPSF (Dickson, K. S., et al., supra, 2001; Dickson, K. S., et al., Mol. Cell. Biol. 19:5707-5717,1999; Mendez, R., et al., Mol. Cell 6:1253-1259, 2000). An additional protein, Cytoplasmic Polyadenylation Element Binding Protein, binds both the RNA and CPSF (Dickson, K. S., et al., supra, 2001; Dickson, K. S., et al., supra, 1999; Mendez, R., et al., supra, 2000), and hence helps to recruit the PAP. RNA-binding proteins, perhaps including CPEB and cytoplasmic CPSF, may be required for the activity of GLD-2 family members as well.

GLD-2, Hs1 and Mm1, each of which are active PAPs, share sequence similarities that distinguish them from canonical nuclear PAPs. In FIG. 11, sequences from the catalytic regions of PAPs are aligned (FIG. 11). All PAPs possess the catalytic (red) and nucleotide binding (green) residues characteristic of β-nucleotidyl transferases. The GLD-2 family members are very closely related to one another (black/grey shading in FIG. 11B), as are the nuclear PAPs (represented by bovine PAP in the aligment (FIG. 11B). The GLD-2 relatives possess deletions and insertions relative to the nuclear PAPs (red asterisks, FIG. 11B). Some (or all) of these features may be required for the activity of this novel form of PAP. However, they are insufficient, since some of these features are also present in proteins that are inactive (e.g., Ce2 and At1; FIG. 11B). At seven positions (green arrowheads, FIG. 11B), each of the enzymes that are active as tethered proteins possess identical or similar amino acids, but differ from those found in the inactive proteins: thus these positions may discriminate PAP enzymes generally. Further tests are needed to determine which features are required for PAP activity or biological functions in vivo, since inactivity in the tethered assay could be due to misfolding or interference by MS2 coat protein.

The discovery of new mouse and human PAPs may provide an entree into new forms of polyadenylation in mammalian cells. GLD-2 regulates stem cell fates in C. elegans; it is required for stem cells to differentiate. Indeed, mutants that lack GLD-2 (and another protein, GLD-1) form germline tumors in which stem cells divide indefinitely (Kadyk, L. C. and Kimble, J., supra, 1998). In S. pombe, Cid1 and Cid13 monitor DNA damage and nucleotide levels (Saitoh, S., supra, 2002; Read, R. L., et al., supra, 2002), thereby controlling the cell cycle. Perhaps, as with GLD-2, Cid1 and Cid13, the mammalian proteins will participate in mitotic controls. In addition, cytoplasmic polyadenylation occurs in mammalian neurons, and likely regulates translation at synapses after they are activated (Job, C. and Eberwine, J., supra, 2001; Martin, K. C., et al., supra, 2000; Richter, J. D., supra, 2000). Transcripts of the new mouse and human PAPs appear to be present in a broad range of tissues, including neurons and stem cells (Stanford Microarray Database). Thus the roles of these enzymes, and of cytoplasmic polyadenylation, should now be accessible in the somatic cells of mice and humans.

### C. Prophetic Methods

i. Specific Metabolic Events. We envision that rPAP activity may be important in the following metabolic events. Thus, we have supplied a description of how one would investigate the role of rPAP.

a. Cancer Chemotherapy

Cells deficient in rPAP activity—such as mutants in genes that we now know encode rPAPs—are dramatically more sensitive to certain valuable chemotherapeutic agents (Walowsky, C., et al., supra, 1999; Wang, S.-W., et al., supra, 2000; Read, R. L., et al., Proc. Natl. Acad. Sci. 99:12079-12084, 2002; Saitoh, S., et al., Cell 109:563-573, 2002). This should make anti-rPAP drugs valuable in combined chemotherapies.

The reasoning hinges on three sets of observations.

Heightened sensitivity in fission yeast. In S. pombe (Wang, S.-W., et al., supra, 2000; Read, R. L., et al., supra, 2002; Saitoh, S., et al., supra, 2002) and in S. cerevisiae (Walowsky, C., et al., supra, 1999), rPAPs are required to cause cell cycle arrest after treatment with certain chemical agents that modify DNA.

Mutants lacking the rPAP are 1000-fold more sensitive to camptothecin and its derivatives, for example. Conversely, S. cerevisiae that over-express an rPAP have reduced sensitivity (Walowsky, C., et al., supra, 1999). Thus the rPAPs are important in governing cessation of the cell cycle in normal cells suffering certain forms of DNA damage.

Camptothecin and cancer. Camptothecin and its derivatives (e.g., CPT-11, Camptostar and Topotecan) have anti-tumor effects in certain human cancers, including colon cancer, ovarian cancer, small cell cancers and brain tumors (e.g., Pommier, Y., et al., supra, 1999; Carmichael, J. and Ozols, R. F., supra, 1997). For example, response rates of 15% or more were seen with patients who had failed first line chemotherapy against ovarian cancer (Carmichael, J. and Ozols, R. F., supra, 1997). These compounds kill tumor cells because they are potent inhibitors of topoisomerase. However, at the doses required, they have serious side effects. Considerable effort is now being expended to develop more efficacious analogs (rev. in Carmichael, J. and Ozols, R. F., supra, 1997).

Genetic links between the rPAPs and human cancer. In humans, a chromosomal region carrying an rPAP homologue is among the most common region amplified in small cell lung tumors and primary small cell tumors (Levin, N. A., et al., Genes Chrom. Can. 13:175-185,1995). This implies that its over-expression may diminish cell cycle control, and so contribute to cancer.

The simplest interpretation of these findings is that the rPAPs regulate an mRNA that is necessary to monitor certain forms of DNA damage and enable cells to repair the insult. As a result, cells that lack the rPAP (or have been exposed to anti-rPAP drugs we hope to find) become hypersensitive to chemotherapeutic agents, including Camptothecin.

To investigate the role of rPAP in chemotherapy, one would preferably examine whether reduction of PAP activity in cultured human tumor cells hypersensitizes those cells to killing by topoisomerase I inhibitors. Reduction in activity will be achieved by: siRNA targeting of the PAP gene; by targeted gene disruption; by overexpression of dominant negative forms of the PAP in vivo (as with enzymes inactivated by mutation at their active sites, e.g., D608 in FIG. 8). The assessment of drug sensitivity in such cells is straightforward to those skilled in the art, as extensive testing of the efficacy of compounds targeted to topoisomerase I (e.g., “tecans”) is underway in several laboratories, and many derivates are already published and in use in the clinic.

b. Regulatory PAPs and Stem Cells

We discovered that rPAPs interact with a family of 3′UTR regulatory proteins, the PUF proteins (Wang, L., et al., supra, 2002; Eckmann, C. R., et al., supra, 2002). Stem cell proliferation—a subject of considerable practical and biological interest—may be the ancestral function of these mRNA regulators (Wang, L., et al., supra, 2002; Wickens, M., et al., Trends in Genet. 18:150-156, 2002; Eckmann, C. R., et al., supra, 2002; Crittenden, S. L., et al., Nature 417:660-664, 2002).

In C. elegans, the rPAP helps maintain the balance of mitosis and differentiation in germline stem cells, by antagonizing PUF protein activity (Wang, L., et al., supra, 2002; Eckmann, C. R., et al., supra, 2002). This function is likely to occur in many species. The mouse and human germlines contain PUF proteins, as do cultured mouse stem cells (Reijo-Pera, R., personal communication). Both also appear to contain rPAP mRNAs (NCBI database). Our hypothesis is that rPAPs support differentiation of stem cells, and that PUF proteins antagonize this role to support proliferation instead (FIG. 7).

rPAPs and PUF proteins control not only conventional stem cells, such as those in mammalian germlines, but may also regulate other continuously dividing populations, such as those of hematopoeitoic or neuronal stem cells (rev in Wickens, M., et al., supra, 2002). In all cases, PUF proteins repress differentiation and sustain proliferation (FIG. 7). We propose that rPAPs do the opposite, enhancing differentiation. Thus agents that affect rPAP activity in stem cell populations might have dramatic affects on stem cell differentiation.

It is striking that mouse ES cells have high levels of the catalytic PAP subunit, as do other cell rich in step cell populations.

To test the role of rPAP in stem cells, one would preferably examine whether reduction of PAP activity in cultured mouse and human stem cells perturbs either their proliferation or their differentiation in response to stimulatory agents. For this purpose, we will use both mouse and human ES cells, neuronal stem cells and hematopoeitoic stem cells. Reduction in PAP activity will be achieved by: siRNA targeting of the PAP gene; by targeted gene disruption; by overexpression of dominant negative forms of the PAP in vivo (as with enzymes inactivated by mutation at their active sites (e.g., D608 in FIG. 8). The assessment of stem cell proliferation and differentiation in response to inducers is obvious to those skilled in the art.

c. Regulatory PAPs and the Nervous System

Cytoplasmic changes in poly(A) length occur not just in the germline, but in somatic cells as well (Muckenthaler, M., et al., RNA 3:983-995,1997; Dehlin, E., et al., Mol. Cell. Biol. 16:468-474,1996; Muschel, R., et al., Mol. Cell. Biol. 6:337-341, 1986; Robinson, B. G., et al., Science 241:342-344,1988; Paek, I. and Axel, R., Mol. Cell. Biol. 7:1496-1507,1987; Rueckert, R., personal communication). Remarkably, the nervous system uses many of the same devices as embryos to move and control their mRNAs (rev. in 7-9, 34-41). Translational control underlies aspects of learning and plasticity (Wells, D. G., et al., supra, 2000; Wu, L., et al., supra, 1998; Quinlan, X., et al., supra, 2001; Richter, J. D., Proc. Natl. Acad. Sci. USA 98:7069-7071, 2001; Krichevsky, A. M. and Kosik, K. S., Neuron 20:683-696, 2001; Job, C. and Eberwine, J., Nat. Rev. Neurosci. 2:889-898, 2001; Barinaga, M., Science 290:736-738, 2000; Barinaga, M., Science 290:737, 2000; Martin, K. C., et al., Curr. Opin. Neurobiol. 10:587-592, 2000; Wells, D. G. and Fallon, J. R., Nat. Neuro. 3:1062-1064, 2000; Mendez, R. and Richter, J. D., supra, 2001). Most importantly, cytoplasmic polyadenylation in particular recently has been linked to learning via translational control at synapses (e.g., Wells, D. G., et al., supra, 2000; Wu, L., et al., supra, 1998; Quinlan, X., et al., supra, 2001; Mendez, R. and Richter, J. D., supra, 2001). The polymerases involved are unknown. We think they are very likely to be the rPAPs we have discovered.

To test whether rPAPs have a role in the nervous system, one would preferably generate transgenic mice and Drosophila lacking the rPAP. Since the rPAP may be essential for viability or development, we will use recombinase systems to selectively eliminate expression in the nervous system. Such systems are well-described for both mice and Drosophila. Conventional assays will be used to assess learning and memory.

ii. Delineation of the Sequence and Structural Features that Define an rPAP

Experimental design. rPAPs are members of a large superfamily of nucleotidyl transferases, including DNA polymerases, enzymes that act on tRNAs, and enzymes that act on small nucleotide analogs. Based on the literature, mere sequence analysis is insufficient to distinguish which is which.

Our tethered function experiments lead to a set of sequences that we know are active as rPAPs, and another set that are not. By compiling and analyzing these sequences, we will identify features (or specific amino acids) that are present in all those that are active and missing from those that are not. FIG. 2d illustrates this strategy, by comparing the rPAP, GLD-2, to the conventional nuclear poly(A) polymerases from several species. The conventional PAPs are all very similar to one another (grey and black boxes), but differ significantly from GLD-2, the rPAP. Moreover, as indicated by red circles and triangles, GLD-2 and the other rPAPs we have identified share certain characteristic differences that we think may be diagnostic.

These analyses enable a prediction of the sequences and features that define an rPAP. We will first test additional candidates to make our predictions more precise. Ultimately, two tests are important:

(1) Certain mutations should inactivate an rPAP, or even convert it into a DNA polymerase instead.

(2) Certain mutations should turn a related DNA polymerase into an rPAP.

We claim:

1. An isolated preparation of a regulatory poly(A) polymerase (PAP), wherein the polymerase comprises both a catalytic subunit and an RNA-binding subunit.

2. An isolated polynucleotide encoding the polymerase of claim 1.

3. The preparation of claim 1 wherein the polymerase comprises GLD-2 and GLD-3 proteins.

4. An isolated polynucleotide encoding the polymerase of claim 3.

5. The preparation of claim 1 wherein the catalytic subunit is GLD-2.

6. The preparation of claim 1 wherein the catalytic subunit is a mutant of GLD-2 that retains catalytic activity.

7. The polymerase of claim 1 wherein the catalytic subunit is hRPAP1 or a mutant of hRPAP that retains catalytic activity.

8. An isolated polynucleotide encoding the polymerase of claim 7.

9. The preparation of claim 1 wherein the RNA-binding subunit is selected from the group consisting of GLD-3, GIP-1 and GIP-2.

10. An isolated polynucleotide encoding the polymerase of claim 9.

11. The polymerase of claim 1 wherein the catalytic subunit is obtained from the mRPAP gene or a mutant of mRPAP that retains catalytic activity.

12. An isolated preparation of the catalytic subunit of an rPAP.

13. The preparation of claim 12 wherein the catalytic subunit is GLD-2 or a mutant of GLD-2 that retains catalytic activity.

14. The preparation of claim 12 wherein the subunit is hRPAP or a mutant that retains catalytic activity.

15. The preparation of claim 12 wherein the catalytic subunit is mRPAP or a mutant that retains catalytic activity.

16. A method of identifying molecules that either increase or decrease the activity of an rPAP, comprising the steps of exposing a candidate molecule to an rPAP and determining whether the candidate molecule increases or decreases polymerase activity.

17. The method of claim 16 wherein the rPAP comprises GLD-2 or a mutant retaining catalytic activity.

18. The method of claim 16 wherein the polymerase is hRPAP or a mutant retaining catalytic activity.

19. The method of claim 18 wherein the catalytic subunit comprises mRPAP or a mutant retaining catalytic activity.

20. The method of claim 16 wherein the catalytic portion of the polymerase is selected from the group consisting of peptides encoded by yeast TRF-5, hRPAP1, and mRPAP.

21. A method of identifying molecules that increase or decrease the activity of the catalytic subunit of an rPAP comprising the step of exposing a candidate molecule to the catalytic subunit of an rPAP and determining whether the candidate molecule increases or decreases polymerase activity.

22. The preparation of claim 21 wherein the subunit comprises GLD-2 or a mutant retaining catalytic activity.

23. The preparation of claim 21 wherein the subunit comprises RPAP or a mutant retaining catalytic activity.

24. The preparation of claim 1 wherein the catalytic subunit comprises mRPAP or a mutant of mRPAP that retains catalytic activity.

25. The polymerase of claim 1 wherein the catalytic subunit is obtained by expression of a gene isolated from H. sapiens.

26. The method of claim 16 wherein the catalytic portion of the polymerase is selected from the group consisting of peptides encoded by yeast TRF-5, hRPAP1, and mRPAP.

27. A method of identifying molecules that are catalytic subunits of rPAPs, comprising the step of comparing the nucleotide or amino acid sequence of the candidate molecule with the GLD-2 sequence and subjecting the corresponding protein to a functional analysis, wherein the candidate protein is a suitable catalytic subunit of an rPAP if the sequence of the protein is related to the GLD-2 sequence and the functional analysis reveals that the protein has poly(A) polymerase activity.

28. A method of adding adenosine residues to an mRNA comprising the step of combining adenosine residues, an mRNA molecule and the polymerase of claim 1, wherein multiple adenosine residues are added to the mRNA.
